(19) United States
(12) Patent Application Publication
(10) Pub. No.: US 2025/0104827 A1
(43) Pub. Date: Mar. 27, 2025

(54) SYSTEMS AND METHODS FOR DETERMINING CANCER THERAPY VIA DEEP LEARNING

(71) Applicant: Artera Inc., Mountain View, CA (US)

(72) Inventors: Andre Esteva, Mountain View, CA (US); Felix Feng, Hillsborough, CA (US); Rikiya Yamashita, Mountain View, CA (US); Siyi Tang, Mountain View, CA (US)

(21) Appl. No.: 18/813,199

(22) Filed: Aug. 23, 2024

Related U.S. Application Data
(63) Continuation of application No. PCT/US2023/013701, filed on Feb. 23, 2023.
(60) Provisional application No. 63/313,505, filed on Feb. 24, 2022.

(51) Int. Cl. G16H 20/10; G16H 50/30

(52) U.S. Cl. CPC G16H 20/10; G16H 50/30

(57) ABSTRACT

The present disclosure provides methods and systems for classifying and/or monitoring a cancer of a subject. A method for assessing a cancer of a subject may comprise obtaining a data set comprising image and/or tabular data from the subject and processing the data with one or more trained algorithms to classify the cancer of the subject. The cancer of the subject may be assessed based on the results of the classification. The assessment may comprise determining a biomarker predictive of a response to a therapeutic intervention for treating the cancer of the subject.

### CROSS-REFERENCE

This application is a continuation of International Application No. PCT/US2023/013701, filed Feb. 23, 2023, which claims the benefit of U.S. Provisional Patent Application No. 63/313,505, filed Feb. 24, 2022, each of which is incorporated by reference herein in its entirety.

### BACKGROUND

Prostate cancer is a leading cause of cancer death in men. Nevertheless, international standards for prognostication of patient outcomes are reliant on non-specific and insensitive tools that commonly lead to over- and under-treatment.

### SUMMARY

Determining a patient's optimal cancer therapy is a challenging task, in which oncologists must choose a therapy with the highest likelihood of success and the least likelihood of toxicity. The difficulties in therapy selection are rooted in the vast molecular, phenotypic, and prognostic heterogeneity exhibited by cancer. Recognized herein is a need for accurate, globally scalable tools to support personalizing cancer therapy.

Radiotherapy is a common form of radical therapy for treatment with curative intent of localized prostate cancer. Improvement in oncologic outcomes has been demonstrated when androgen deprivation therapy (ADT) is added to radiotherapy. However, ADT has well documented toxicity, including hot flashes, declines in libido and erectile function, loss of muscle mass, increase in body fat, decreased bone density, and potential deleterious effects on cardiac and brain health.

Despite the benefits of ADT, a majority of men with localized prostate cancer treated with radiotherapy alone without ADT never develop distant metastasis. Unfortunately, there remain no predictive biomarkers to identify which men specifically derive benefit from ADT with radiotherapy, and thus current guidelines recommend the use of ADT based on prognosis, estimated using National Comprehensive Cancer Network (NCCN) risk groups. Thus, there is a large unmet need to guide the use of ADT with radiotherapy for men with localized prostate cancer.

Accordingly, disclosed herein are methods and systems for identifying or monitoring cancer-related states by processing biological samples obtained from or derived from subjects, e.g., a cancer patient. Biological samples (e.g., tissue samples) obtained from subjects may be analyzed to prognose clinical outcomes (which may include, e.g., distant metastasis, biochemical recurrence, death, progression free survival, and overall survival). Biological samples obtained from subjects may be analyzed to predict response to treatment and guide treatment decisions.

In an aspect, the present disclosure provides a method for assessing a cancer of a subject, comprising: (a) obtaining a dataset comprising at least image data obtained or derived from the subject; (b) processing the dataset using a trained algorithm to classify the dataset to a category among a plurality of categories, wherein the classifying comprises applying an image processing algorithm to the image data; and (c) assessing the cancer based at least in part on classification of the dataset to the category, wherein the assessing comprises determining a biomarker predictive of a response to a therapeutic intervention for treating the cancer of the subject.

In some embodiments, the response comprises overall survival. In some embodiments, the response comprises progression free survival. In some embodiments, the response comprises reduction in mortality. In some embodiments, the response comprises reduction in prostate cancer-specific mortality. In some embodiments, the response comprises metastasis-free survival. In some embodiments, the response comprises reduction in metastasis. In some embodiments, the response comprises reduction in distant metastasis. In some embodiments, the response comprises reduction in distant metastasis at five years.

In some embodiments, the method further comprises determining whether the subject is biomarker positive or biomarker negative for the biomarker, wherein the determining whether the subject is biomarker positive or biomarker negative comprises: (i) calculating a first probability of the subject displaying the response in a presence of the therapeutic intervention; (ii) calculating a second probability of the subject displaying the response in an absence of the therapeutic intervention; (iii) calculating a probability delta between the first probability and the second probability; and (iv) comparing the probability delta to a reference standard.

In some embodiments, the method further comprises determining whether the subject is biomarker positive or biomarker negative for the biomarker, wherein the determining whether the subject is biomarker positive or biomarker negative comprises: (i) calculating a first probability of the subject displaying the response in a presence of the therapeutic intervention and in a presence of radiation therapy (RT); (ii) calculating a second probability of the subject displaying the response in an absence of the therapeutic intervention and in a presence of radiation therapy (RT); (iii) calculating a probability delta between the first probability and the second probability; and (iv) comparing the probability delta to a reference standard. In some embodiments, the subject is biomarker positive when the probability delta is higher than the reference standard; and the subject is negative for the biomarker when the probability delta is lower than the reference standard. In some embodiments, the reference standard is determined at least in part by measuring a median probability delta from a plurality of subjects.

In some embodiments, the method further comprises (i) calculating a first probability of the subject displaying the response in the presence of the therapeutic intervention and in the presence of radiation therapy (RT); (ii) calculating a second probability of the subject displaying the response in the absence of the therapeutic intervention and in the presence of radiation therapy (RT); (iii) calculating a probability delta between the first probability and the second probability; and (iv) comparing the probability delta to a reference standard; wherein the subject is biomarker positive when the probability delta is higher than the reference standard; and wherein the subject is negative for the biomarker when the probability delta is lower than the reference standard. In some embodiments, the subject is biomarker positive when the probability delta is higher than the reference standard; and the subject is negative for the biomarker when the probability delta is lower than the reference standard. In some embodiments, the reference standard is determined at least in part by measuring a median probability delta from a plurality of subjects.

In some embodiments, the biomarker positive subject is a candidate for the therapeutic intervention. In some embodiments, the method further comprises treating the subject with the therapeutic intervention. In some embodiments, the therapeutic intervention comprises androgen deprivation therapy (ADT). In some embodiments, the ADT is short-term ADT (ST-ADT). In some embodiments, the trained algorithm is trained using self-supervised learning. In some embodiments, the trained algorithm comprises a deep learning algorithm. In some embodiments, the dataset further comprises tabular data. In some embodiments, the trained algorithm comprises a first trained algorithm processing the image data and a second trained algorithm processing the tabular data. In some embodiments, the trained algorithm further comprises a third trained algorithm processing outputs of the first and second trained algorithm. In some embodiments, the tabular data comprises clinical data of the subject. In some embodiments, the clinical data comprises laboratory data, therapeutic interventions, or long-term outcomes. In some embodiments, the cancer comprises prostate cancer, bladder cancer, breast cancer, pancreatic cancer, or thyroid cancer. In some embodiments, the cancer comprises prostate cancer. In some embodiments, the image data comprises digital histopathology data. In some embodiments, the data comprise images derived from a biopsy sample of the subject. In some embodiments, the images are acquired via microscopy of the biopsy sample. In some embodiments, the method further comprises processing the image data using an image segmentation, image concatenation, or object detection algorithm. In some embodiments, the method further comprises extracting a feature from the image data.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a computer system that is programmed or otherwise configured to implement methods provided herein.

FIGS. 2A-2C show an example of a multimodal deep learning system and dataset. FIG. 2A shows that the multi-modal architecture comprises three parts: a tower stack to parse the tabular clinical data, a tower stack to parse a variable number of digital histopathology slides, and a third tower stack to merge the resultant features and predict binary outcomes. FIG. 2B shows the training of the self-supervised model of the image tower stack. FIG. 2C illustrates a schematic representation of a multimodal AI (MMAI) system. The example MMAI system illustrated in FIG. 2C accepts both tabular (e.g., clinical) data and image (e.g., histopathology) data and outputs a delta score that characterizes a magnitude of therapeutic benefit to a patient.

FIGS. 3A-3H show an example of a comparison of the deep learning system to established clinical guidelines across trials and outcomes. FIG. 3A shows performance results reporting on the area under the curve (AUC) of sensitivity and specificity of the MMAI (blue bars) vs NCCN (gray bars) models, using time-dependent receiver operator characteristics. Comparison is made across 5- and 10-year timepoints on the following binary outcomes: distant metastasis (DM), biochemical recurrence (BCR), prostate cancer-specific survival (PCaSS), and overall survival (OS). FIG. 3B shows a summary table of the relative improvement of the AI model over the NCCN model across the various outcomes and broken down by performance on the data from each trial in the test set. Relative improvement is given by (PAI−PNCCN)/PNCCN, where P is the performance of a model. FIG. 3C shows the results of an ablation study showing model performance when trained on a sequentially decreasing set of data inputs. NCCN means the following three variables: combined Gleason, baseline psa, t-stage; NCCN+3 means NCCN plus: Gleason primary, Gleason secondary, age; path refers to digitized histopathology images. FIGS. 3D-3H show a performance comparison on the individual clinical trial subsets of the test set-together, these five comprise the entire test set shown in FIG. 3A.

FIG. 4 shows an example of pathologist interpretation of SSL tissue clusters. The self-supervised model in the multi-modal model is trained to identify whether or not augmented versions of small patches of tissue come from the same original patch, without ever seeing clinical data labels. After training, each image patch in the dataset of 10.05M image patches is fed through this model to extract a 128-dimensional feature vector, and the UMAP algorithm31 is used to cluster and visualize the resultant vectors. A pathologist is then asked to interpret the 20 image patches closest to each of the 25 cluster centroids—the descriptions are shown next to the insets. For clarity, we only highlight 6 clusters (colored), and show the remaining clusters in gray. See FIG. 7 for full pathologist annotation.

FIG. 5 shows an example of image quilts for four example patients. The dataset contains patients with a variable number of histopathology slides. To standardize the image inputs to the model, the tissue from each slide is segmented, and all tissues are pasted into a single square image of 51200×51200 pixels and divided into 200 by 200 patches, representing all the histopathology data of a single patient. Image quilts from four patients are shown here.

FIG. 6 shows an example of nucleic density sampling of example image patches. Tan brown boxes indicate nuclei detection, which is used for calculating nucleic density. We oversample the patches that are inputted to the self-supervised training protocol according to nucleic density. Each patch is binned into deciles according to density, and each decile is oversampled such that the MMAI model sees the same number of total images from each decile.

FIG. 7 shows an example of pathologist-interpreted patch clusters. Using UMAP, 25 clusters are generated from the SSL features of all the histopathology patches of trial RTOG-9202. Each row in the image corresponds to the 25 nearest-neighbor image patches of the cluster centroid. These have been inspected by a pathologist to determine the human-interpretable descriptions of the clusters listed in the table.

FIG. 8 shows an example of an NCCN model algorithm. A rule-based algorithm modeling the National Cancer Center Network's annual published guidelines, based on the D'Amico risk groups.

FIG. 9 is a table showing statistics from processed clinical trial data. The first five columns list statistics for each trial. The column ‘combined’ shows the statistics of the final dataset with all five trials used for training and validation. ***RTOG 9413 randomized patients in a 2×2 fashion testing the effect of timing of hormone therapy (before vs. starting with RT) and field size (prostate only vs. full pelvic RT). New acronyms utilized: disease-free survival (DFS), progression-free survival (PFS), prostate cancer-specific mortality (PCSM).

FIG. 10 illustrates a CONSORT flow diagram of processing a clinical dataset. ST-ADT=short-term androgen deprivation therapy; RT=radiation therapy.

FIG. 11 illustrates relative feature importance of various tabular (e.g., clinical) and image (e.g., histopathology) data features as determined using a trained algorithm. The feature importance was calculated based on the mean of the absolute Shapely value for each variable and normalized across features. Image importance measured 37.3%, followed by Gleason Primary: 35.8%, Gleeson Secondary: 9.0%, Gleason Combined: 5.6%, T-stage: 5.5%, Age: 3.5%, and Baseline PSA: 3.4%.

FIG. 12 illustrates distributions of delta scores for development (left) and validation (right) cohorts as determined using a trained algorithm. The vertical lines indicated the 67th percentile which was chosen as the boundary between “biomarker positive” and “biomarker negative” subjects.

FIG. 13 illustrates cumulative incidence curves for distant metastasis by AI-biomarker subgroups (“biomarker positive” and “biomarker negative”) as determined using a trained algorithm.

FIG. 14 illustrates forest plots for distant metastasis (DM) and prostate cancer-specific mortality (PCSM) across positive and negative biomarker groups.

FIG. 15 illustrates forest plots for distant metastasis (DM) and prostate cancer-specific mortality (PCSM) in positive and negative biomarker groups for the subgroup of National Comprehensive Cancer Network (NCCN) low-intermediate-risk patients.

FIG. 16 illustrates cumulative incidence curves for prostate cancer-specific mortality (PCSM) by AI-biomarker subgroups (“biomarker positive” and “biomarker negative”) as determined using a trained algorithm.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, an individual, or a patient. A subject can be a vertebrate, such as, for example, a mammal. Non-limiting examples of mammals include humans, simians, farm animals, sport animals, rodents, and pets. A subject can be a male subject. A subject can be a female subject. The subject may be displaying a symptom(s) indicative of a health or physiological state or condition of the subject, such as a cancer-related health or physiological state or condition of the subject. As an alternative, the subject can be asymptomatic with respect to such health or physiological state or condition. The subject may be suspected of having a health or physiological state or condition. The subject may be at risk of developing a health or physiological state or condition. The health or physiological state may correspond to a disease (e.g., cancer). The subject may be an individual diagnosed with a disease. The subject may be an individual at risk of developing a disease.

As used herein, “diagnosis of cancer,” “diagnosing cancer,” and related or derived terms include the identification of cancer in a subject, determining the malignancy of the cancer, or determining the stage of the cancer.

As used herein, “prognosis of cancer,” “prognosing cancer,” and related or derived terms include predicting the clinical outcome of the patient, assessing the risk of cancer recurrence, determining treatment modality, or determining treatment efficacy.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

### Embodiments of the Disclosure

Despite the prevalence of prostate cancer, accurate, sensitive, and specific diagnosis of prostate cancer remains elusive. While prostate cancer is often indolent, and treatment can be curative, prostate cancer represents the leading global cause of cancer-associated disability due to the negative effects of over- and under-treatment and remains one of the leading causes of cancer death in men. Determining the optimal course of therapy for patients with prostate cancer is a difficult medical task that involves considering the patient's overall health, the characteristics of their cancer, the side effect profiles of many possible treatments, outcomes data from clinical trials involving patients with similar diagnoses and prognosticating the expected future outcomes of the patient at hand. This challenge is compounded by the lack of readily accessible prognostic tools to better risk stratify patients.

Artificial intelligence (AI) has permitted insights to be gleaned from massive datasets that had previously resisted interpretation. Whereas standard risk-stratification tools are fixed and based on few variables, AI can learn from large amounts of minimally processed data across various modalities. AI systems may be low-cost, massively scalable, and incrementally improve through usage.

There is a great need for accurate, globally scalable tools to support personalizing therapy. Methods and systems as disclosed herein demonstrate prostate cancer therapy personalization by predicting the impact of specific therapies on long-term, clinically relevant outcomes (e.g., distant metastasis, biochemical recurrence, partial response, complete response, death, relative survival, cancer-specific survival, progression free survival, disease free survival, five-year survival, and overall survival) using a novel multimodal deep learning model trained on digital histopathology of prostate biopsies and clinical data.

Accordingly, the present disclosure provides methods, systems, and kits for identifying or monitoring cancer-related categories and/or states by processing biological samples obtained from or derived from subjects (e.g., male patients suffering from or suspected of suffering from prostate cancer). Biological samples (e.g., prostate biopsy samples) obtained from subjects may be analyzed to identify the cancer-related category (which may include, e.g., measuring a presence, absence, or quantitative assessment (e.g., risk, predicted outcome) of the cancer-related category or measuring a predicted or observed response to a therapeutic intervention). Such subjects may include subjects with one or more cancer-related categories and subjects without cancer-related categories. Cancer-related categories or states may include, for example, positive for a cancer, negative for a cancer, cancer stage, observed response to a cancer treatment (e.g., radiotherapy, chemotherapy, surgical intervention), observed long-term outcome (e.g., disease metastasis, biochemical recurrence, partial response, complete response, relative survival, cancer-specific survival, progression free survival, disease free survival, five-year survival, or overall survival), predicted response to a cancer treatment, and/or predicted long-term outcome.

### Assaying Biological Samples

A biological sample may be obtained or derived from a human subject (e.g., a male subject). The biological sample may be stored in a variety of storage conditions before processing, such as different temperatures (e.g., at room temperature, under refrigeration or freezer conditions, at 25° C., at 4° C., at −18° C., −20° C., or at −80° C.), different suspensions (e.g., formalin, EDTA collection tubes, cell-free RNA collection tubes, or cell-free DNA collection tubes), or. The biological sample may be obtained from a subject having or suspected of having cancer (e.g., prostate cancer), or from a subject that does not have or is not suspected of having cancer.

A biological sample may be used for diagnosing, detecting or identifying a disease or health or physiological condition of a subject by analyzing the biological sample. The biological sample or part thereof may be analyzed to determine a likelihood the sample is positive for a disease or health condition (e.g., prostate cancer). Alternatively or additionally, methods as described herein may include diagnosing a subject with the disease or health condition, monitoring the disease or health condition in the subject, and/or determining a propensity of the subject for the health disease/condition. In some embodiments, the biological sample(s) may be used to classify the sample and/or subject into a cancer-related category and/or identify the subject as having a particular cancer-related state. The cancer-related category or state may comprise a diagnosis (e.g., positive or negative for cancer), a particular type of cancer (e.g., prostate cancer), a stage of cancer, a predicted outcome or prognosis, a predicted response to a treatment or treatments, or any combination thereof.

Any substance that is measurable may be the source of a sample. The substance may be a fluid, e.g., a biological fluid. A fluidic substance may include blood (e.g., whole blood, plasma, serum), cord blood, saliva, urine, sweat, serum, semen, vaginal fluid, gastric and digestive fluid, cerebrospinal fluid, placental fluid, cavity fluid, ocular fluid, serum, breast milk, lymphatic fluid, or combinations thereof.

The substance may be solid, for example, a biological tissue. The substance may comprise normal healthy tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The substance may comprise a tumor. Tumors may be benign (non-cancer), pre-malignant, or malignant (cancer), or any metastases thereof. Non-limiting examples of tumors and associated cancers may include: acoustic neuroma, acute lymphoblastic leukemia, acute myeloid leukemia, adenocarcinoma, adrenocortical carcinoma, AIDS-related cancers, AIDS-related lymphoma, anal cancer, angiosarcoma, appendix cancer, astrocytoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, bronchogenic carcinoma, cerebellar astrocytoma, cervical cancer, childhood cancers, chondrosarcoma, chordoma, choriocarcinoma, chronic lymphocytic leukemia, chronic myelogenous leukemia, chronic myeloproliferative disorders, colon cancer, colon carcinoma, craniopharyngioma, cutaneous T-cell lymphoma, cystadenocarcinoma, desmoplastic small round cell tumor, embryonal carcinoma, endocrine system carcinomas, endometrial cancer, endotheliosarcoma, ependymoma, epithelial carcinoma, esophageal cancer, Ewing's sarcoma, fibrosarcoma, germ cell tumors, gallbladder cancer, gastric cancer, gastrointestinal carcinoid tumor, gastrointestinal stromal tumor, gastrointestinal system carcinomas, genitourinary system carcinomas, gliomas, hairy cell leukemia, head and neck cancer, heart cancer, hemangioblastoma, hepatocellular (liver) cancer, Hodgkin lymphoma, Hypopharyngeal cancer, intraocular melanoma, islet cell carcinoma, Kaposi sarcoma, kidney cancer, laryngeal cancer, leiomyosarcoma, lip and oral cavity cancer, liposarcoma, liver cancer, lung cancers, such as non-small cell and small cell lung cancer, lung carcinoma, lymphangiosarcoma, lymphangioendotheliosarcoma, lymphomas, leukemias, macroglobulinemia, malignant fibrous histiocytoma of bone/osteosarcoma, medulloblastoma, medullary carcinoma, melanomas, meningioma, mesothelioma, metastatic squamous neck cancer with occult primary, mouth cancer, multiple endocrine neoplasia syndrome, myelodysplastic syndromes, myeloid leukemia, myxosarcoma, nasal cavity and paranasal sinus cancer, nasopharyngeal carcinoma, neuroblastoma, non-Hodgkin lymphoma, non-small cell lung cancer, oligodendroma, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, papillary adenocarcinoma, papillary carcinoma, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, sebaceous gland carcinoma, seminoma, skin cancers, skin carcinoma merkel cell, small intestine cancer, soft tissue sarcoma, squamous cell carcinoma, stomach cancer, sweat gland carcinoma, synovioma, T-cell lymphoma, testicular tumor, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, Wilms tumor, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The substances may comprise a mix of normal healthy tissues or tumor tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof. In some embodiments, the tissues are associated with a prostate of the subject. In the case of a biological sample comprising cells and/or tissue (e.g., a biopsy sample), the biological sample may be further analyzed or assayed. In some embodiments, the biopsy sample may be fixed, processed (e.g., dehydrated), embedded, frozen, stained, and/or examined under a microscope. In some embodiments, digital slides are generated from processed samples.

In some embodiments, the substance may comprise a variety of cells, including: eukaryotic cells, prokaryotic cells, fungi cells, heart cells, lung cells, kidney cells, liver cells, pancreas cells, reproductive cells, stem cells, induced pluripotent stem cells, gastrointestinal cells, blood cells, cancer cells, bacterial cells, bacterial cells isolated from a human microbiome sample, and circulating cells in the human blood. In some embodiments, the substance may comprise contents of a cell, such as, for example, the contents of a single cell or the contents of multiple cells.

In some embodiments, the substances may comprise one or more markers whose presence or absence is indicative of some phenomenon such as disease, disorder, infection, or environmental exposure. A marker can be, for example, a cell, a small molecule, a macromolecule, a protein, a glycoprotein, a carbohydrate, a sugar, a polypeptide, a nucleic acid (e.g., deoxyribonucleic acid (DNA), ribonucleic acid (RNA)), a cell-free nucleic acid (e.g., cf-DNA, cf-RNA), a lipid, a cellular component, or combinations thereof.

The biological sample may be taken before and/or after treatment of a subject with cancer. Biological samples may be obtained from a subject during a treatment or a treatment regimen. Multiple biological samples may be obtained from a subject to monitor the effects of the treatment over time. The biological sample may be taken from a subject known or suspected of having a cancer (e.g., prostate cancer). The biological sample may be taken from a subject experiencing unexplained symptoms, such as fatigue, nausea, weight loss, aches and pains, weakness, or bleeding. The biological sample may be taken from a subject having explained symptoms. The biological sample may be taken from a subject at risk of developing cancer due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

After obtaining a biological sample from the subject, the biological sample may be processed to generate datasets indicative of a disease, condition, cancer-related category, or health state of the subject. For example, a tissue sample may be subjected to a histopathological assay (e.g., microscopy, including digital image acquisition such as whole slide imaging) to generate image data based on the biological sample. Alternatively, a liquid sample or a marker isolated from a sample may be subject to testing (e.g., a clinical laboratory test) to generate tabular data. In some embodiments, a sample is assayed for the presence, absence, or amount of one or more metabolites (e.g., prostate specific antigen (PSA)).

### Types of Data

Methods and systems as described herein make take as inputs one or more datasets. The one or more datasets may comprise tabular and/or image data. The tabular and/or image data may be derived from a biological sample of the subject. In some embodiments, the data are not derived form a biological sample.

The data may comprise images of tissue samples taken from a biopsy of a subject. The image data may be acquired by microscopy of the biopsy sample. The microscopy may comprise optical microscopy, virtual or digital microscopy (such as whole slide imaging (WSI)), or any suitable microscopy technique known in the field. The microscopy images may be subjected to one or more processing steps such as filtering, segmentation, concatenation, or object detection.

Tabular data as described herein may comprise any non-image data relevant to a health state or condition (e.g., disease) of a subject. Tabular data may comprise clinical data such as laboratory data at one or more timepoints (e.g., prostate serum antigen (PSA) level), qualitative measures of cell pathology (e.g., Gleason grade, Gleason score), structured or unstructured health data (e.g., digital rectal exam results), medical imaging data or results (e.g., results of an x-ray, computed tomography (CT) scan, magnetic resonance imaging (MRI) scan, positron-emission tomography (PET) scan, or ultrasound, such as transrectal ultrasound results), age, medical history, previous or current cancer state (e.g., remission, metastasis) or stage, current or previous therapeutic interventions, long-term outcome, and/or National Comprehensive Cancer Network (NCCN) classification or its constituents (e.g., combined Gleason score, t-stage, baseline PSA).

In some embodiments, the therapeutic intervention may comprise radiotherapy (RT). In some embodiments, the therapeutic intervention may comprise chemotherapy. In some embodiments, the therapeutic intervention may comprise a surgical intervention. In some embodiments, the therapeutic intervention may comprise an immunotherapy. In some embodiments, the therapeutic intervention may comprise a hormone therapy. In some embodiments, the RT may comprise RT with pre-specified use of short-term androgen deprivation therapy (ST-ADT). In some embodiments, the RT may comprise RT with pre-specified use of long-term ADT (LT-ADT). In some embodiments, the RT may comprise RT with pre-specified use of dose escalated RT (DE-RT). In some embodiments, the surgical intervention may comprise radical prostatectomy (RP). In some embodiments, the therapeutic intervention may comprise any combination of therapeutic interventions disclosed herein. In some embodiments, the long-term outcome may comprise distant metastasis (DM). In some embodiments, the long-term outcome may comprise biochemical recurrence (BR). In some embodiments, the long-term outcome may comprise partial response. In some embodiments, the long-term outcome may comprise complete response. In some embodiments, the long-term outcome may comprise death. In some embodiments, the long-term outcome may comprise relative survival. In some embodiments, the long-term outcome may comprise cancer-specific survival. In some embodiments, the cancer-specific survival may comprise prostate cancer-specific survival (PCaSS). In some embodiments, the long-term outcome may comprise progression free survival. In some embodiments, the long-term outcome may comprise disease free survival. In some embodiments, the long-term outcome may comprise five-year survival. In some embodiments, the long-term outcome may comprise overall survival (OS). In some embodiments, the long-term outcome may comprise any combination of long-term outcomes disclosed herein.

Data as used in methods and systems described herein may be subject to one or more processing steps. In some embodiments, data (e.g., image data) is subjected to an image processing, image segmentation, and/or object detection process as encoded in an image processing, image segmenting, or object detection algorithm. The image processing procedure may filter, transform, scale, rotate, mirror, shear, combine, compress, segment, concatenate, extract features from, and/or smooth an image prior to downstream processing. In some embodiments, a plurality of images (e.g., histopathology slides) is combined to form an image quilt. The image quilt may be converted to a representation (e.g., a tensor) that is useful for downstream processing of image data. The image segmentation process may partition an image into one or more segments which contain a factor or region of interest. For example, an image segmentation algorithm may process digital histopathology slides to determine a region of tissue as opposed to a region of whitespace or an artifact. In some embodiments, the image segmentation algorithm may comprise a machine learning or artificial intelligence algorithm. In some embodiments, image segmentation may precede image processing. In some embodiments, image processing may precede image segmentation. The object detection process may comprise detecting the presence or absence of a target object (e.g., a cell or cell part, such as a nucleus). In some embodiments, object detection may proceed image processing and/or image segmentation. For example, images which are found by an image detection algorithm to contain one or more objects of interest may be concatenated in a subsequent image processing step. Alternatively or additionally, image processing may precede object detection and/or image segmentation. For example, raw image data may be processed (e.g., filtered) and the processed image data subjected to an object detection algorithm. Image data may be subject to multiple image processing, image segmentation, and/or object detection steps in any appropriate order. In an example, image data is optionally subjected to one or more image processing steps to improve image quality. The processed image is then subjected to an image segmentation algorithm to detect regions of interest (e.g., regions of tissue in a set of histopathology slides). The regions of interest are then subjected to an object detection algorithm (e.g., an algorithm to detect nuclei in images of tissue) and regions found to possess at least one target object are concatenated to produce processed image data for downstream use.

In some embodiments, data (e.g., tabular data) may be subject to one or more processing steps. Processing steps may include, without limitation, standardization or normalization. The one or more processing steps may, for example, discard data which contain spurious values or contain very few observations. The one or more processing steps may further or alternatively standardize the encoding of data values. Different input datasets may have the same parameter value encoded in different ways, depending on the source of the dataset. For example, ‘900’, ‘900.0’, ‘904’, ‘904.0’, ‘−1’, ‘−1.0’, ‘None’, and ‘NaN’ may all encode for a “missing” parameter value. The one or more processing steps may recognize the encoding variation for the same value and standardize the dataset to have a uniform encoding for a given parameter value. The processing step may thus reduce irregularities in the input data for downstream use. The one or more data sets may normalize parameter values. For example, numerical data may be scaled, whitened, colored, decorrelated, or standardized. For example, data may be scaled or shifted to lie in a particular interval (e.g., [0,1] or [−1, 1]) and/or have correlations removed. In some embodiments, categorical data may be encoded as a one-hot vector. In some embodiments, one or more different types of tabular (e.g., numerical, categorical) data may be concatenated together. In some embodiments, data is not subjected to a processing step.

Data may be taken at one or more timepoints. In some embodiments, data is taken at an initial timepoint and a later timepoint. The initial timepoint and the later timepoint may be spaced by any appropriate amount of time, such as 1 hour, 1 day, 1 week, 2 weeks, 3 weeks, 4 weeks, 6 weeks, 12 weeks, 4 months, 5 months, 6 months, 7 months, 8 months, 9 months, 10 months, 11 months, 1 years, 2 years, 3 years, 4 years, 5 years, 6 years, 7 years, 8 years, 9 years, 10 years, or more. In some embodiments, the data is from more than two timepoints. In some embodiments, the data are from 3, 4, 5, 6, 7, 8, 9, 10, 11, 12, 13, 14, 15, 16, 17, 18, 19, 20, or more timepoints.

### Trained Algorithms

After using one or more assays to process one or more biological samples derived from the subject to generate one or more datasets indicative of the cancer state (e.g., cancer-related category or categories) of the subject, a trained algorithm may be used to process one or more of the datasets (e.g., a visual data and/or tabular data) to determine a cancer state of the subject. For example, the trained algorithm may be used to determine the presence or absence of (e.g., prostate) cancer in the subject based on the image data and/or laboratory data. The trained algorithm may be configured to identify the cancer state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise an unsupervised machine learning algorithm. The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm.

In some embodiments, a machine learning algorithm of a method or system as described herein utilizes one or more neural networks. In some case, a neural network is a type of computational system that can learn the relationships between an input dataset and a target dataset. A neural network may be a software representation of a human neural system (e.g. cognitive system), intended to capture “learning” and “generalization” abilities as used by a human. In some embodiments, the machine learning algorithm comprises a neural network comprising a CNN. Non-limiting examples of structural components of machine learning algorithms described herein include: CNNs, recurrent neural networks, dilated CNNs, fully-connected neural networks, deep generative models, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network (DNN)) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation, e.g., a summation operation. A connection from an input to a node is associated with a weight (or weighting factor). The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

The number of nodes used in the input layer of the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater. In other instances, the number of node used in the input layer may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less. In some instances, the total number of layers used in the ANN or DNN (including input and output layers) may be at least about 3, 4, 5, 10, 15, 20, or greater. In other instances, the total number of layers may be at most about 20, 15, 10, 5, 4, 3, or less.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater. In other instances, the number of learnable parameters may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less.

In some embodiments of a machine learning algorithm as described herein, a machine learning algorithm comprises a neural network such as a deep CNN. In some embodiments in which a CNN is used, the network is constructed with any number of convolutional layers, dilated layers or fully-connected layers. In some embodiments, the number of convolutional layers is between 1-10 and the dilated layers between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or less, and the total number of dilated layers may be at most about 20, 15, 10, 5, 4, 3, or less. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or less.

In some embodiments, a machine learning algorithm comprises a neural network comprising a CNN, RNN, dilated CNN, fully-connected neural networks, deep generative models and/or deep restricted Boltzmann machines.

In some embodiments, a machine learning algorithm comprises one or more CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully-connected layers and normalization layers. The layers may be organized in 3 dimensions: width, height and depth.

The convolutional layers may apply a convolution operation to the input and pass results of the convolution operation to the next layer. For processing images, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the width and height of the input volume, compute the dot product between the entries of the filter and the input, and produce a two-dimensional activation map of that filter. As a result, the network may learn filters that activate when it detects some specific type of feature at some spatial position in the input.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers connect every neuron in one layer to every neuron in another layer. In neural networks, each neuron may receive input from some number locations in the previous layer. In a fully-connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

The trained algorithm may be configured to accept a plurality of input variables and to produce one or more output values based on the plurality of input variables. The plurality of input variables may comprise one or more datasets indicative of a cancer-related category. For example, an input variable may comprise a microscopy image of a biopsy sample of the subject. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the biological sample and/or the subject by the classifier. The trained algorithm may comprise a binary classifier, such that each of the one or more output values comprises one of two values (e.g., {0, 1}, {positive, negative}, or {high-risk, low-risk}) indicating a classification of the biological sample and/or subject by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample and/or subject by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's cancer-related category, and may comprise, for example, a therapeutic intervention, a duration of the therapeutic intervention, and/or a dosage of the therapeutic intervention suitable to treat a subject classified in a particular cancer-related category.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the cancer-related category of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a cancer-related state (e.g., type or stage of cancer) or belonging to a cancer-related category. For example, a binary classification of samples may assign an output value of “negative” or 0 if the sample indicates that the subject has less than a 50% probability of belonging to a cancer-related category. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject belongs to a cancer-related category (e.g., cancer diagnosis or prognosis) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of belonging to cancer-related category (e.g., long-term outcome) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a cancer-related state or belonging to a cancer-related category (e.g., positive for prostate cancer) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a cancer-related state (e.g., for prostate cancer) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated datasets obtained by assaying the biological sample (as described elsewhere herein), clinical data form the subject, and one or more known output values corresponding to the biological sample and/or subject (e.g., a clinical diagnosis, prognosis, absence, or treatment efficacy of a cancer-related state of the subject). Independent training samples may comprise biological samples and associated datasets and outputs obtained or derived from a plurality of different subjects. Independent training samples may comprise biological samples and associated datasets and outputs obtained at a plurality of different time points from the same subject (e.g., on a regular basis such as weekly, biweekly, monthly, annually, etc.). Independent training samples may be associated with presence of the cancer-related state (e.g., training samples comprising biological samples and associated datasets and outputs obtained or derived from a plurality of subjects known to have the cancer-related state). Independent training samples may be associated with absence of the cancer-related state (e.g., training samples comprising biological samples and associated datasets and outputs obtained or derived from a plurality of subjects who are known to not have a previous diagnosis of the cancer-related state or who have received a negative test result for the cancer-related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples and clinical data associated with presence of the cancer-related category and/or cell-free biological samples and clinical data associated with absence of the cancer-related category. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the cancer-related category. In some embodiments, the biological sample and/or clinical data is independent of samples used to train the trained algorithm.

The trained algorithm may be trained with a first number of independent training samples associated with presence of the cancer-related category and a second number of independent training samples associated with absence of the cancer-related category. The first number of independent training samples associated with presence of the cancer-related category may be no more than the second number of independent training samples associated with absence of the cancer-related category. The first number of independent training samples associated with presence of the cancer-related category may be equal to the second number of independent training samples associated with absence of the cancer-related category. The first number of independent training samples associated with presence of the cancer-related category may be greater than the second number of independent training samples associated with absence of the cancer-related category.

Various loss functions can be used to train the algorithm. In some embodiments, the algorithm may comprise a regression loss function. In some embodiments, the algorithm may comprise a logistic loss function. In some embodiments, the algorithm may comprise a cross-entropy loss function. In some embodiments, the algorithm may comprise a (e.g., negative) log-likelihood loss functions. In some embodiments, the algorithm may comprise a negative partial log-likelihood loss. In some embodiments, the algorithm may comprise a variational loss. In. In some embodiments, the loss functions may be formulated to optimize a regression loss, an evidence-based lower bound, a maximum likelihood, Kullback-Leibler divergence, applied with various distribution functions such as Gaussians, non-Gaussian, mixtures of Gaussians, mixtures of logistic functions, and so on.

In some embodiments, an algorithm as described herein is trained in a multitask manner. In such cases, the algorithm may be trained to perform multiple learning tasks at the same time. In an example, an algorithm is trained to perform a first classification task (e.g., associating subject data with a cancer related category). The algorithm is further trained to perform a second task comprising associating the subject data with a second cancer-related category. In some cases, the algorithm is further trained to calculate a delta or deviation between the likelihood of the first and second classifications. The first and second classifications may correspond to any cancer-related categories as described herein. The first and second classifications may be based on the same subject data. Alternatively, they may be based on different or transformed subject data. In an example, a first classification is based on factual data associated with one or more therapeutic interventions a subject received while the second classification is based on counterfactual data associated with one or more therapeutic interventions the subject could receive.

Various optimizers can be used to train the neural network. In some embodiments, the neural network may be trained with the Adam optimizer. In some embodiments, the neural network may be trained with the stochastic gradient descent optimizer. In some embodiments, the neural network may be trained with an active learning algorithm. A neural network may be trained with various loss functions whose derivatives may be computed to update one or more parameters of the neural network. A neural network may be trained with hyperparameter searching algorithms. In some embodiments, the neural network hyperparameters are optimized with Gaussian Processes.

The trained algorithm may be configured to identify the cancer-related category at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the cancer-related category by the trained algorithm may be calculated as the percentage of independent test samples (e.g., subjects known to belong to the cancer-related category or subjects with negative clinical test results for the cancer-related category) that are correctly identified or classified as belonging to or not belonging to the cancer-related category.

The trained algorithm may be configured to identify the cancer-related category with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the cancer-related category using the trained algorithm may be calculated as the percentage of cell-free biological samples identified or classified as having the cancer-related category that correspond to subjects that truly belong to the cancer-related category.

The trained algorithm may be configured to identify the cancer-related category with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the cancer-related category using the trained algorithm may be calculated as the percentage of subject datasets identified or classified as not having the cancer-related category that correspond to subjects that truly do not belong to the cancer-related category.

The trained algorithm may be configured to identify the cancer-related category with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the cancer-related category using the trained algorithm may be calculated as the percentage of independent test samples associated with the cancer-related category (e.g., subjects known to belong to the cancer-related category) that are correctly identified or classified as having the cancer-related category.

The trained algorithm may be configured to identify the cancer-related category with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the cancer-related category using the trained algorithm may be calculated as the percentage of independent test samples associated with absence of the cancer-related category (e.g., subjects with negative clinical test results for the cancer-related category) that are correctly identified or classified as not belonging to the cancer-related category.

The trained algorithm may be configured to identify the cancer-related category with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying datasets derived from a subject as belonging to or not belonging to the cancer-related category.

The trained algorithm may be adjusted or tuned to improve one or more of the performance, accuracy, PPV, NPV, clinical sensitivity, clinical specificity, or AUC of identifying the cancer-related category. The trained algorithm may be adjusted or tuned by adjusting parameters of the trained algorithm (e.g., a set of cutoff values used to classify a biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the clinical data may be identified as most influential or most important to be included for making high-quality classifications or identifications of cancer-related categories (or sub-types of cancer-related categories). The clinical data or a subset thereof may be ranked based on classification metrics indicative of each parameter's influence or importance toward making high-quality classifications or identifications of cancer-related categories (or sub-types of cancer-related categories). Such metrics may be used to reduce, in some embodiments significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

Systems and methods as described herein may use more than trained algorithm to determine an output (e.g., cancer-related category of a subject). Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., image data or tabular data). Alternatively, a trained algorithm may be trained on more than one type of data. The inputs of one trained algorithm may comprise the outputs of one or more other trained algorithms. Additionally, a trained algorithm may receive as its input the output of one or more trained algorithms.

### Identifying or Monitoring a Cancer-Related Category or State

After using a trained algorithm to process the dataset, the cancer-related category or may be identified or monitored in the subject. The identification may be based at least in part on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites.

The cancer-related category may characterize a cancer-related state of the subject. By way of nonlimiting example, the cancer related state may comprise a subject having or not having a cancer (e.g., prostate cancer), a subject being at risk or having a risk level (e.g., high risk, low risk) for a cancer, a predicted long-term outcome of a cancer (e.g., distant metastasis, biochemical recurrence, partial response, complete response, overall survival, cancer-specific survival, progression free survival, disease free survival, five-year survival, death), response or receptiveness to a therapeutic intervention, or any combination thereof.

The subject may be identified as belonging to a cancer-related category at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the cancer-related category of the individual by the trained algorithm may be calculated as the percentage of independent test samples (e.g., subjects known to belong to the cancer-related category or subjects with negative clinical test results corresponding to the cancer-related category) that are correctly identified or classified as belonging to or not belonging to the cancer-related category.

The subject may be determined as belonging to a cancer-related category with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the cancer-related category using the trained algorithm may be calculated as the percentage of biological samples identified or classified as belonging to the cancer-related category that correspond to subjects that truly belong to the cancer-related category.

The cancer-related category may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the cancer-related category using the trained algorithm may be calculated as the percentage of biological samples identified or classified as not having the cancer-related category that correspond to subjects that truly do not have the cancer-related category.

The subject may be identified as belonging to the cancer-related category with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the cancer-related category using the trained algorithm may be calculated as the percentage of independent test samples associated with belonging to the cancer-related category (e.g., subjects known to belong to the cancer-related category) that are correctly identified or classified as belonging to the cancer-related category.

The cancer-related category may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the cancer-related category using the trained algorithm may be calculated as the percentage of independent test samples associated with not belonging to the cancer-related category (e.g., subjects with negative clinical test results for the cancer-related category) that are correctly identified or classified as not belonging to the cancer-related category.

After the cancer-related category is identified in a subject, a sub-type of the cancer-related category (e.g., selected from among a plurality of sub-types of the cancer-related category) may further be identified. The sub-type of the cancer-related category may be determined based at least in part on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites. For example, the subject may be identified as being at risk of a sub-type of prostate cancer (e.g., from among a number of sub-types of prostate cancer). After identifying the subject as being at risk of a sub-type of prostate cancer, a clinical intervention for the subject may be selected based at least in part on the sub-type of prostate cancer for which the subject is identified as being at risk. In some embodiments, the clinical intervention is selected from a plurality of clinical interventions (e.g., clinically indicated for different sub-types of prostate cancer).

Upon identifying the subject as belonging to the cancer-related category, the subject may be optionally provided with a therapeutic intervention (e.g., prescribing an appropriate course of treatment to treat the type, sub-type, or state of the cancer of the subject). The therapeutic intervention may comprise a prescription of an effective dose of a drug or other therapy (e.g., radiotherapy, chemotherapy), a surgical intervention (e.g., radical prostatectomy), a further testing or evaluation of the cancer-related category, a further monitoring of the cancer-related category, or a combination thereof. If the subject is currently being treated for the cancer-related category with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

The analysis of biopsy samples (e.g., analysis of microscopy images of prostate tissue), proteomic data comprising quantitative measures of proteins of the dataset at a panel of cancer-related category-associated proteins, and/or metabolome data comprising quantitative measures of a panel of cancer-related category-associated metabolites may be assessed over a duration of time to monitor a patient (e.g., subject who has or is at risk for cancer or who is being treated for a cancer). In such cases, the measures of the dataset of the patient may change during the course of treatment. For example, the measures of the dataset of a patient with decreasing risk of the cancer-related category due to an effective treatment may shift toward the profile or distribution of a healthy subject (e.g., a subject without a cancer or in remission from cancer). Conversely, for example, the measures of the dataset of a patient with increasing risk of the cancer-related category due to an ineffective treatment may shift toward the profile or distribution of a subject with higher risk of the cancer-related category or a more advanced cancer-related category.

The cancer-related category of the subject may be monitored by monitoring a course of treatment for treating the cancer or cancer-related state of the subject. The monitoring may comprise assessing the cancer-related category or state of the subject at two or more time points. The assessing may be based at least on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined at each of the two or more time points.

In some embodiments, a difference in quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of one or more clinical indications, such as (i) a diagnosis of the cancer-related state of the subject, (ii) a prognosis of the cancer-related state of the subject, (iii) an increased risk of the cancer-related state of the subject, (iv) a decreased risk of the cancer-related state of the subject, (v) an efficacy of the course of treatment for treating the cancer-related state of the subject, and (vi) a non-efficacy of the course of treatment for treating the cancer-related state of the subject.

In some embodiments, a difference in quantitative or qualitative measures of biological samples (e.g., of histopathology slides of biopsy samples), proteomic data comprising quantitative measures of proteins of the dataset at a panel of cancer-related category-associated proteins, and/or metabolome data comprising quantitative measures of a panel of cancer-associated metabolites determined between the two or more time points may be indicative of a diagnosis of the cancer-related state or category of the subject. For example, if the cancer-related state was not detected in the subject at an earlier time point but was detected in the subject at a later time point, then the difference is indicative of a diagnosis of the cancer-related state of the subject. A clinical action or decision may be made based on this indication of diagnosis of the cancer-related state of the subject, such as, for example, prescribing a new therapeutic intervention for the subject. The clinical action or decision may comprise recommending the subject for a secondary clinical test to confirm the diagnosis of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of a prognosis of the cancer-related category of the subject.

In some embodiments, a difference in the quantitative or qualitative measures of biological samples (e.g., of histopathology slides of biopsy samples), proteomic data comprising quantitative measures of proteins of the dataset at a panel of cancer-related category-associated proteins, and/or metabolome data comprising quantitative measures of a panel of cancer-associated metabolites determined between the two or more time points may be indicative of the subject having an increased risk of the cancer-related state. For example, if the cancer-related state was detected in the subject both at an earlier time point and at a later time point, and if the difference is a negative difference (e.g., the quantitative or qualitative measures of biological samples (e.g., of histopathology slides of biopsy samples), proteomic data comprising quantitative measures of proteins of the dataset at a panel of cancer-related category-associated proteins, and/or metabolome data comprising quantitative measures of a panel of cancer-related category-associated metabolites increased from the earlier time point to the later time point), then the difference may be indicative of the subject having an increased risk of the cancer-related state. A clinical action or decision may be made based on this indication of the increased risk of the cancer-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of the subject having a decreased risk of the cancer-related state. For example, if the cancer-related state was detected in the subject both at an earlier time point and at a later time point, and if the difference is a positive difference (e.g., the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites decreased from the earlier time point to the later time point), then the difference may be indicative of the subject having a decreased risk of the cancer-related state. A clinical action or decision may be made based on this indication of the decreased risk of the cancer-related state (e.g., continuing or ending a current therapeutic intervention) for the subject. The clinical action or decision may comprise recommending the subject for a secondary clinical test to confirm the decreased risk of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of an efficacy of the course of treatment for treating the cancer-related state of the subject. For example, if the cancer-related state was detected in the subject at an earlier time point but was not detected in the subject at a later time point, then the difference may be indicative of an efficacy of the course of treatment for treating the cancer-related state of the subject. A clinical action or decision may be made based on this indication of the efficacy of the course of treatment for treating the cancer-related state of the subject, e.g., continuing or ending a current therapeutic intervention for the subject. The clinical action or decision may comprise recommending the subject for a secondary clinical test to confirm the efficacy of the course of treatment for treating the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of a non-efficacy of the course of treatment for treating the cancer-related category of the subject. For example, if the cancer-related state was detected in the subject both at an earlier time point and at a later time point, and if the difference is a negative or zero difference (e.g., the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites increased or remained at a constant level from the earlier time point to the later time point), and if an efficacious treatment was indicated at an earlier time point, then the difference may be indicative of a non-efficacy of the course of treatment for treating the cancer-related state of the subject. A clinical action or decision may be made based on this indication of the non-efficacy of the course of treatment for treating the cancer-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the cancer-related state. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

### Predicting a Response to a Therapeutic Intervention

After using a trained algorithm to process the dataset, the cancer-related category or may be identified or monitored in the subject. The identification may be based at least in part on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites. In some cases, the cancer-related category may comprise a predicted response to a therapeutic intervention.

The predicted response may be determined by processing data (e.g., tabular and/or image data) associated with the subject in a factual model. Under a factual model associated the subject, the data represent a therapeutic intervention that a subject actually did or did not receive. For example, a factual model for a patient that received androgen deprivation therapy (ADT) as (e.g., at least part of) a therapeutic intervention may comprise an indication that the subject has been treated with ADT. In another example, a factual model for a patient that has not received ADT as (e.g., at least part of a therapeutic intervention) may comprise an indication that the subject has not received ADT. The predicted response may be further determined by again processing the data, or a subset thereof, associated with the subject except under a counterfactual model which indicates the opposite with respect to administration of the therapy to the subject. For example, a counterfactual model for a patient that received ADT as (e.g., at least part of) a therapeutic intervention may comprise an indication that the subject has not been treated with ADT while a corresponding counterfactual model for a subject that has not received ADT as (e.g., at least part of) a therapeutic intervention may comprise an indication that the subject did receive ADT. By processing both a factual and counterfactual model, an observed or predicted benefit of the therapeutic intervention may then be calculated.

The difference in the likelihood of the predicted cancer-related state (e.g., long term outcome, such as distant metastasis [DM] or cancer-type-specific mortality) as predicted from the factual and counterfactual models may be described as a delta score or treatment delta.

In some cases, trained algorithms as described herein may be configured to determine a predicted benefit of the therapeutic intervention by predicting the treatment delta for a subject. The trained algorithm may further be trained on a delta loss as described herein. The delta loss can characterize the deviation between the predicted delta scores and the expected delta scores for a given training subject.

The expected delta scores may depend on a subgroup of the training subject given their treatment types and long-term outcome (e.g., DM).

In some cases, a subgroup comprises subjects who did not receive the therapeutic intervention and do not display the cancer-related category (e.g., are negative for DM). In such cases, the treatment delta is expected to be 0 or close to 0 as the subjects did not display the cancer-related category in the absence of the therapeutic intervention (e.g., subjects who did not receive the therapeutic intervention nevertheless did not develop distant metastasis as may be expected in the absence of treatment).

In some cases, a subgroup comprise subjects who did not receive the therapeutic intervention and display the cancer-related category (e.g., are positive for DM). In such cases, the treatment delta is expected to be greater than or equal to 0 since the subjects may benefit from the therapeutic intervention (e.g., the subjects who did not received the therapeutic intervention may have benefited from having had it).

In some cases, a subgroup comprises subjects who received the therapeutic intervention and are negative for the cancer-related category. In such cases, the treatment delta is expected to be greater than 0 since the subjects were negative for the cancer-related category in presence of the therapeutic intervention (e.g., subjects who received the therapeutic intervention showed reduced DM, indicating efficacy of the intervention).

In some cases, a subgroup comprises subjects who received the therapeutic intervention and are positive for the cancer-related category. In such cases, the treatment delta is expected to be 0 or close to 0 since the subjects are positive for the cancer-related category even in the presence of the therapeutic intervention (e.g., subjects who received the therapeutic intervention nevertheless developed DM, indicating inefficacy of the treatment). During training, the model may be penalized when delta scores do not fall in the expected range.

Following training of the algorithm, the trained algorithm may be used to predict a response to a therapeutic intervention for previously unseen subjects. In some cases, the trained algorithm may predict a subject is positively indicated (“positive” or “biomarker positive”) for the therapeutic intervention if the subject is predicted to show a reduced risk for the cancer related category in the presence of the therapeutic intervention. The classification of the subject as biomarker positive may occur if the predicted treatment delta for the subject is above a cutoff. The cutoff may comprise a value, such as great than about −0.5, −0.4, −0.3, −0.2, −0.1, 0, 0.1, 0.2, 0.3, 0.4, 0.5, 0.6, 0.7, 0.8, or 0.9, or more. The cutoff may be determined based on a population of subjects. In an example, the cutoff may be selected based on a percentile of a distribution of treatment deltas observed across a population of subjects. The cutoff may correspond to about the 10th, 20th, 30th, 40th, 50th, 60th, 70th, 80th, or 90th, or more, percentile. The cutoff may correspond to at least about the 10th, 20th, 30th, 40th, 50th, 60th, 70th, 80th, or 90th, or more, percentile. In some cases, the cutoff may correspond to at most about the 90th, 80th, 70th, 60th, 50th, 40th, 30th, 20th, or 10th, or less, percentile. In some cases, the cutoff is between any two of these values.

In some embodiments, the therapeutic intervention may comprise radiotherapy (RT). In some embodiments, the therapeutic intervention may comprise chemotherapy. In some embodiments, the therapeutic intervention may comprise a surgical intervention. In some embodiments, the therapeutic intervention may comprise an immunotherapy. In some embodiments, the therapeutic intervention may comprise a hormone therapy. In some embodiments, the RT may comprise RT with pre-specified use of short-term androgen deprivation therapy (ST-ADT). In some embodiments, the RT may comprise RT with pre-specified use of long-term ADT (LT-ADT). In some embodiments, the RT may comprise RT with pre-specified use of dose escalated RT (DE-RT). In some embodiments, the surgical intervention may comprise radical prostatectomy (RP). In some embodiments, the therapeutic intervention may comprise any combination of therapeutic interventions disclosed herein. In some embodiments, the long-term outcome may comprise distant metastasis (DM). In some embodiments, the long-term outcome may comprise biochemical recurrence (BR). In some embodiments, the long-term outcome may comprise partial response. In some embodiments, the long-term outcome may comprise complete response. In some embodiments, the long-term outcome may comprise death. In some embodiments, the long-term outcome may comprise relative survival. In some embodiments, the long-term outcome may comprise cancer-specific survival. In some embodiments, the cancer-specific survival may comprise prostate cancer-specific survival (PCaSS). In some embodiments, the long-term outcome may comprise progression free survival. In some embodiments, the long-term outcome may comprise disease free survival. In some embodiments, the long-term outcome may comprise five-year survival. In some embodiments, the long-term outcome may comprise overall survival (OS). In some embodiments, the long-term outcome may comprise any combination of long-term outcomes disclosed herein.

In some embodiments, the therapeutic intervention comprises an additional therapeutic intervention. In an example, the therapeutic intervention comprises hormone therapy in addition to radiotherapy. In some cases, the hormone therapy is androgen deprivation therapy (ADT). In some cases, ADT is short term-ADT (ST-ADT). In some cases, the ADT is long-term ADT (LT-ADT).

In some embodiments, the subject may be “biomarker positive” if the subject is predicted to show a reduced risk of DM if treated with the therapeutic intervention. In some embodiments, the subject may be biomarker positive if the subject is predicted to show a reduced risk of BR if treated with the therapeutic intervention. In some embodiments, the subject may be biomarker positive if the subject is predicted to show a reduced risk of cancer type-specific mortality (such as prostate cancer-specific mortality) if treated with the therapeutic intervention. In some embodiments, the subject may be biomarker positive if the subject is predicted to show a reduced risk of death if treated with the therapeutic intervention. In some embodiments, the subject may be biomarker positive if the subject is predicted to show an increased likelihood of partial response. In some embodiments, the subject may be biomarker positive if the subject is predicted to show an increased likelihood of complete response. In some embodiments, the subject may be biomarker positive if the subject is predicted to show an increased likelihood of overall survival. In some embodiments, the subject may be biomarker positive if the subject is predicted to show an increased likelihood of five-year survival. In some embodiments, the subject may be biomarker positive if the subject is predicted to show an increased likelihood of ten-year survival. In some embodiments, the subject may be biomarker positive if the subject is predicted to show an increased likelihood of fifteen-year survival.

In some embodiments, the subject may be “biomarker negative” if the subject is predicted not to show a decreased risk (e.g., no change or increased risk) of DM if treated with the therapeutic intervention. In some embodiments, the subject may be biomarker negative if the subject is predicted not to show a reduced risk of BR if treated with the therapeutic intervention. In some embodiments, the subject may be biomarker negative if the subject is predicted not to show a reduced risk of cancer type-specific mortality (such as prostate cancer-specific mortality) if treated with the therapeutic intervention. In some embodiments, the subject may be biomarker negative if the subject is predicted not to show a reduced risk of death if treated with the therapeutic intervention. In some embodiments, the subject may be biomarker negative if the subject is predicted not to show an increased likelihood of partial response. In some embodiments, the subject may be biomarker negative if the subject is predicted to show an increased likelihood of complete response. In some embodiments, the subject may be biomarker negative if the subject is predicted not to show an increased likelihood of overall survival. In some embodiments, the subject may be biomarker negative if the subject is predicted not to show an increased likelihood of five-year survival. In some embodiments, the subject may be biomarker negative if the subject is predicted not to show an increased likelihood of ten-year survival. In some embodiments, the subject may be biomarker negative if the subject is predicted not to show an increased likelihood of fifteen-year survival.

### Outputting a Report of the Cancer-Related State

After the cancer-related state is identified or an increased risk of the cancer-related state is monitored in the subject, a report may be electronically outputted that is indicative of (e.g., identifies or provides an indication of) the cancer-related state of the subject. The subject may not display a cancer-related state (e.g., is asymptomatic of the cancer-related state such as a presence or risk of prostate cancer). The report may be presented on a graphical user interface (GUI) of an electronic device of a user. The user may be the subject, a caretaker, a physician, a nurse, or another health care worker.

The report may include one or more clinical indications such as (i) a diagnosis of the cancer-related state of the subject, (ii) a prognosis of the cancer-related category of the subject, (iii) an increased risk of the cancer-related category of the subject, (iv) a decreased risk of the cancer-related category of the subject, (v) an efficacy of the course of treatment for treating the cancer-related category of the subject, (vi) a non-efficacy of the course of treatment for treating the cancer-related category of the subject, and (vii) a long-term outcome of the cancer-related category. The report may include one or more clinical actions or decisions made based on these one or more clinical indications. Such clinical actions or decisions may be directed to therapeutic interventions or further clinical assessment or testing of the cancer-related state of the subject.

For example, a clinical indication of a diagnosis of the cancer-related state of the subject may be accompanied with a clinical action of prescribing a new therapeutic intervention for the subject. As another example, a clinical indication of an increased risk of the cancer-related state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the cancer-related state of the subject may be accompanied with a clinical action of continuing or ending a current therapeutic intervention for the subject. As another example, a clinical indication of an efficacy of the course of treatment for treating the cancer-related state of the subject may be accompanied with a clinical action of continuing or ending a current therapeutic intervention for the subject. As another example, a clinical indication of a non-efficacy of the course of treatment for treating the cancer-related state of the subject may be accompanied with a clinical action of ending a current therapeutic intervention and/or switching to (e.g., prescribing) a different new therapeutic intervention for the subject.

### Computer Systems

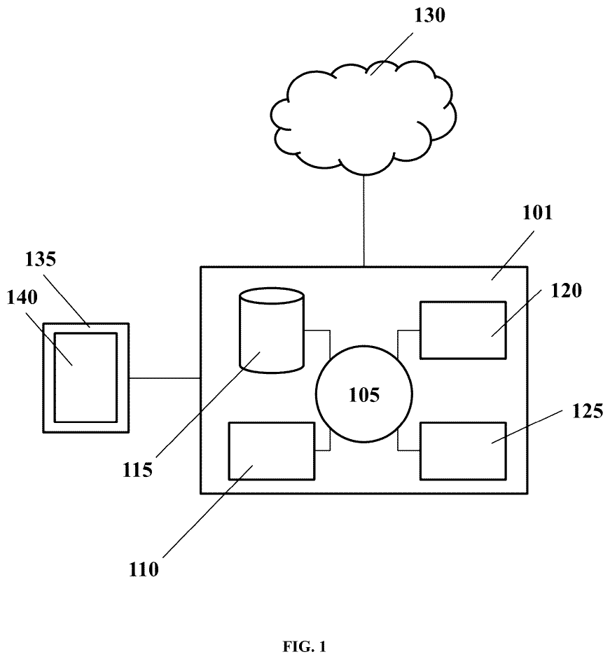

The present disclosure provides computer systems that are programmed to implement methods of the disclosure. FIG. 1 shows a computer system 101 that is programmed or otherwise configured to, for example, (i) train and test a trained algorithm, (ii) use the trained algorithm to process image and/or tabular data to determine a cancer-related category or cancer-related state of a subject, (iii) assess a cancer of the subject based on a classified category, (iv) identify or monitor the cancer-related category or state of the subject, and (v) electronically output a report that indicative of the cancer-related category or state of the subject.

The computer system 101 can regulate various aspects of analysis, calculation, and generation of the present disclosure, such as, for example, (i) training and testing a trained algorithm, (ii) using the trained algorithm to process image and/or tabular data to determine a cancer-related category or cancer-related state of a subject, (iii) assessing a cancer of the subject based on a classified category, (iv) identifying or monitoring the cancer-related category or state of the subject, and (v) electronically outputting a report that indicative of the cancer-related category or state of the subject. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet.

In some embodiments, the network 130 is a telecommunication and/or data network. The network 130 can include one or more computer servers, which can enable distributed computing, such as cloud computing. For example, one or more computer servers may enable cloud computing over the network 130 (“the cloud”) to perform various aspects of analysis, calculation, and generation of the present disclosure, such as, for example, (i) training and testing a trained algorithm, (ii) using the trained algorithm to process data to determine a cancer-related category of a subject, (iii) determining a quantitative measure indicative of a cancer-related category of a subject, (iv) identifying or monitoring the cancer-related category of the subject, and (v) electronically outputting a report that indicative of the cancer-related category of the subject. Such cloud computing may be provided by cloud computing platforms such as, for example, Amazon Web Services (AWS), Microsoft Azure, Google Cloud Platform, and IBM cloud. In some embodiments, the network 130, with the aid of the computer system 101, can implement a peer-to-peer network, which may enable devices coupled to the computer system 101 to behave as a client or a server.

The CPU 105 may comprise one or more computer processors and/or one or more graphics processing units (GPUs). The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 115 can store files, such as drivers, libraries and saved programs. The storage unit 115 can store user data, e.g., user preferences and user programs. In some embodiments, the computer system 101 can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine-readable code can be provided in the form of software. During use, the code can be executed by the processor 105. In some embodiments, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Embodiments of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, (i) a visual display indicative of training and testing of a trained algorithm, (ii) a visual display of data indicative of a cancer-related category of a subject, (iii) a quantitative measure of a cancer-related category of a subject, (iv) an identification of a subject as having a cancer-related category, or (v) an electronic report indicative of the cancer-related category of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, (i) train and test a trained algorithm, (ii) use the trained algorithm to process image and/or tabular data to determine a cancer-related category or cancer-related state of a subject, (iii) assess a cancer of the subject based on a classified category, (iv) identify or monitor the cancer-related category or state of the subject, and (v) electronically output a report that indicative of the cancer-related category or state of the subject.

### EXAMPLES

### Example 1: Prostate Cancer Therapy Personalization Via Multi-Modal Deep Learning

Methods and systems as disclosed herein demonstrate prostate cancer therapy personalization by predicting long-term, clinically relevant outcomes (distant metastasis, biochemical recurrence, death from prostate cancer, and overall survival) using a novel multimodal deep learning model trained on digital histopathology of prostate biopsies and clinical data. An example system of the present disclosure comprises a trained algorithm that was trained and validated using a dataset of five phase III randomized multinational trials run across hundreds of clinical sites. Clinical and histopathological data was available for 5,654 of 7,957 patients (71.1%), which yielded 16.1 terabytes of histopathology imagery, with 10-20 years of patient follow-up. Compared to the most commonly used risk stratification tool, the National Cancer Center Network's (NCCN) risk groups, the deep learning model had superior prognostic and discriminatory performance across all tested outcomes. This artificial intelligence system may allow oncologists to computationally model the likeliest outcomes of any specific patient and thus determine their optimal treatment. Outfitted with digital histopathology scanners and internet access, any clinic could offer such capabilities, enabling low-cost universal access to vital therapy personalization.

The NCCN risk groups are based on the international standards for risk stratification, developed in the late 1990s and referred to as the D'Amico risk groups. This system is based on a digital rectal exam, a serum prostate-specific antigen (PSA) measurement, and the grade of a tumor assessed by histopathology. This three-tier system continues to form the backbone of treatment recommendations throughout the world but has suboptimal prognostic and discriminatory performance to risk stratify patients. This in part is due to the highly subjective and non-specific nature of the core variables in these models. For instance, Gleason grading was developed in the 1960s and remains highly subjective, with unacceptable interobserver reproducibility even amongst expert urologic pathologists. More recently, tissue-based genomic biomarkers have demonstrated improved prognostic performance. However, nearly all of these tests lack validation in prospective randomized clinical trials in the intended use population, and there has been little to no international adoption due to costs and processing time. As such, there remains a serious unmet clinical need for improved tools to personalize therapy for prostate cancer.

Artificial intelligence (AI) has demonstrated remarkable capabilities across a number of use-cases in medicine, ranging from physician-level diagnostics to workflow optimization, and has the potential to support cancer therapy as clinical adoption of digital histopathology continues. AI has begun making progress in histopathology-based prognostics, for instance by predicting short-term patient outcomes or by improving the accuracy of Gleason-based cancer grading on postoperative surgical samples. Whereas standard risk-stratification tools are fixed and based on few variables, AI can learn from large amounts of minimally processed data across various modalities. In contrast to genomic biomarkers, AI systems are low-cost, massively scalable, and incrementally improve through usage. Furthermore, a key challenge for any biomarker is having optimal data to train and validate the relevant endpoint(s), and some commercial prognostic biomarkers in oncology may have been trained on retrospective convenience sampling.

Methods and systems as disclosed herein may comprise a multimodal artificial intelligence (MMAI) system that can meaningfully overcome the unmet need for outcomes prognostication in localized prostate cancer, creating a generalizable biomarker with the potential for global adoption. Prognostic biomarkers localized prostate cancer using five phase III randomized clinical trials were used to train an algorithm as described herein by leveraging multi-modal deep learning on digital histopathology.

A unique dataset from five large multi-national randomized phase III clinical trials of men with localized prostate cancer (NRG/RTOG 9202, 9408, 9413, 9910, and 0126) was generated. All patients received definitive radiotherapy (RT), with pre-specified use of short-term androgen deprivation therapy (ST-ADT), long-term ADT (LT-ADT), and/or dose-escalated RT (DE-RT) (FIG. 9). Of the 7,957 patients enrolled in these five trials, there were 7,752 patients with complete baseline clinical data and 5,654 with complete baseline and digital histopathology data. This represents 16.1 TB of histopathology imagery from 16,204 histopathology slides of pretreatment biopsy samples.

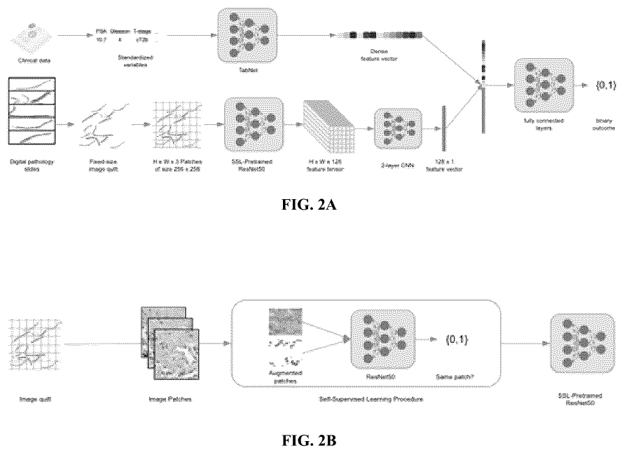

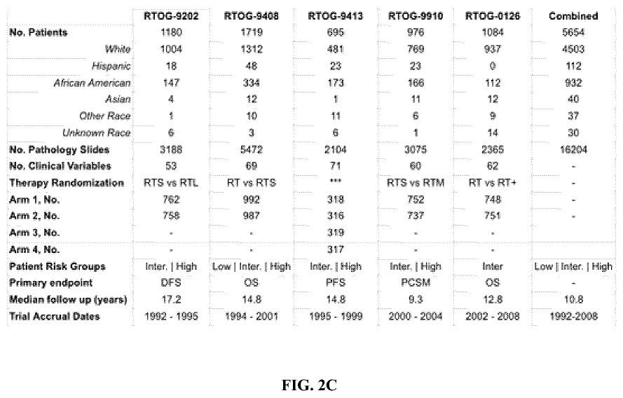

The MMAI architecture can ingest both tabular (clinical) and image-based (histopathology) data, making it uniquely suited for randomized clinical trial data. The full architecture is shown in FIG. 2A. Each patient in the dataset is represented by clinical variables—including laboratory and pathology data, therapeutic interventions, and long-term outcomes—and digitized histopathology slides (median of 3.5 slides). Joint learning across both data streams is complex and involves building three separate deep learning pipelines-one for the imagery, one for the tabular data, and a third to unite them. The data were standardized across the trials for consistency.

Effective learning of relevant features from a variable number of digitized histopathology slides involved several pre-processing steps to standardize the images, followed by self-supervised training. For each patient, all the tissue sections in their biopsy slides were segmented out and combined into a single large image, called an image quilt (FIG. 5), of a fixed width and height across all patients. An H×W grid was overlain on the image quilt which cut it into patches of size 256×256 pixels across its RGB channels. These patches were then used to train a self-supervised (SSL) model to learn histopathological features useful for downstream AI tasks. FIG. 2B shows this part of the pipeline. Once trained, the SSL model could then take the patches of an image quilt and output a 128-dimensional vector representation for each patch. Concatenating all of these vectors in the same spatial orientation as the original patches yielded an H×W×128 tensor (a feature-quilt) that compressed the initially massive image quilt into a compact representation useful for further downstream learning.

SSL is a method that may be used for learning from datasets without annotations. Typical ML setups leverage supervised learning, in which datasets are composed of data points (e.g., images) and data labels (e.g., object classes). In contrast, during SSL, synthetic data labels are extracted from the original data and used to train generic feature representations which can be used for downstream tasks. Momentum contrast—a technique which takes the set of image patches, generates augmented copies of each patch, then trains a model to predict whether any two augmented copies come from the same original patch—may be effective at learning features for medical tasks. The structural setup is shown in FIG. 2B, with further details described elsewhere herein.

To guide the SSL process towards patch regions that are likely to be more clinically useful, patches in the dataset were oversampled based on nucleic density. Using an object detection model trained to detect nuclei the number of nuclei in each patch was approximated. The patches were binned into deciles based on this count, and each decile was oversampled such that the net number of images seen during one epoch of training is the same for each decile. Examples images are shown in FIG. 6.

Systems as described herein may learn from patient-level annotations with the histopathology slides left unannotated. Moreover, the self-supervised learning of the image model allows it to learn from new image data without the need for any annotations.

Learning from the tabular data comprised two steps. First, the clinical data was standardized across the trials and used to pre-train a TabNet architecture via self-supervision by masking parts of data and training the model to learn them. Then, each patient's data was fed through TabNet to extract a feature vector, which was then concatenated with the output of the image pipeline. The concatenated vector was then fed through further neural layers and the model output a binary outcome for the task at hand.

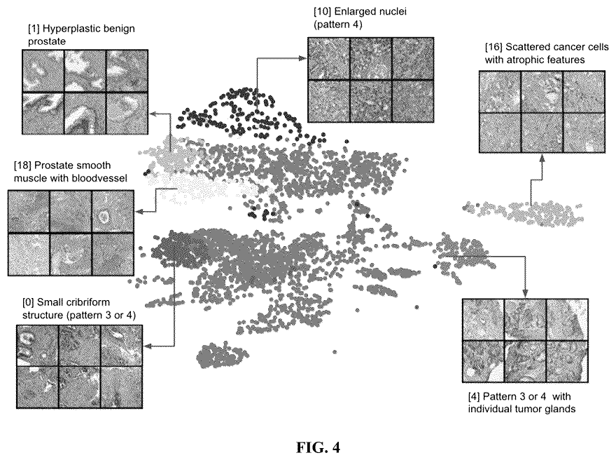

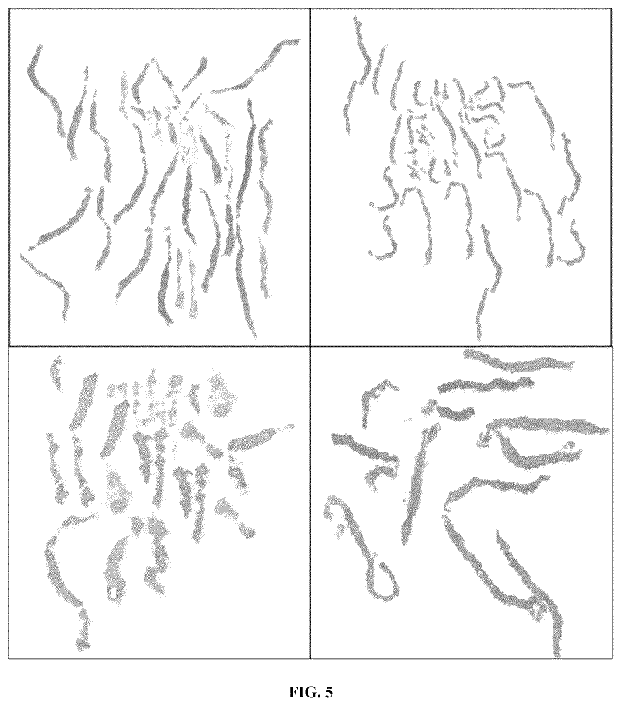

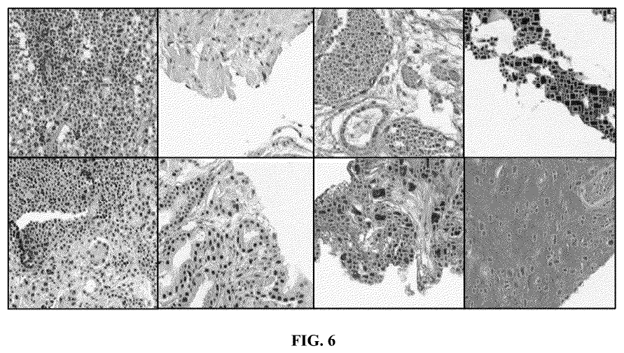

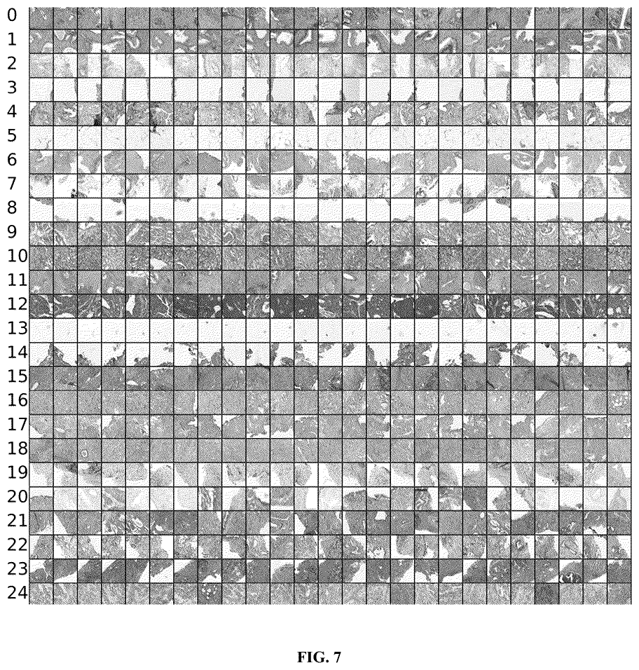

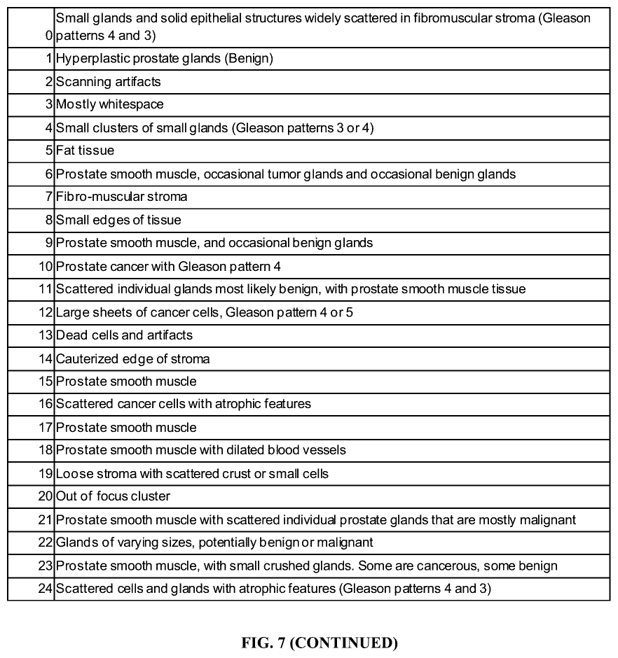

The internal data representations of the SSL model are shown in FIG. 4. The entire dataset's image patches were fed through the SSL model and model features—a 128-dimensional vector outputted by the model—were extracted for each patch. Uniform Manifold Approximation and Projection algorithm (UMAP) was then applied to these features, projecting them from 128 dimensions down to two, and each patch was plot as an individual point. Neighboring data points represent image patches that the model considered similar. UMAP grouped the feature vectors into 25 clusters, some of which are shown in various colors. Insets show example image patches that are close in feature space to cluster centroids. The 20 nearest-neighbor image patches to the cluster centroids were then interpreted by a pathologist. Example interpretations are shown in FIG. 4, and the full interpretation is shown in FIG. 7. The SSL model learned human-interpretable image features that are indicative of complex aspects of cancer, such as Gleason grade or tissue type, despite never being trained with clinical annotations.

Six different MMAI models were trained and tested across four endpoints (DM, BCR, PCaSS, OS) and two timeframes: 5-year and 10-year. The performance of these models was measured with the area under the time-dependent receiver operator characteristic curve (AUC) of sensitivity and specificity, accounting for competing events. Sensitivity is defined as the ratio of correct positive predictions to positive events shown (sensitivity=predicted_positive/num_positive), and the specificity is defined as the ratio of correct negative predictions to negative events shown (specificity=predicted_negative/num_negative). For this metric, 0.5 denotes random chance accuracy and 1.0 denotes perfect accuracy.

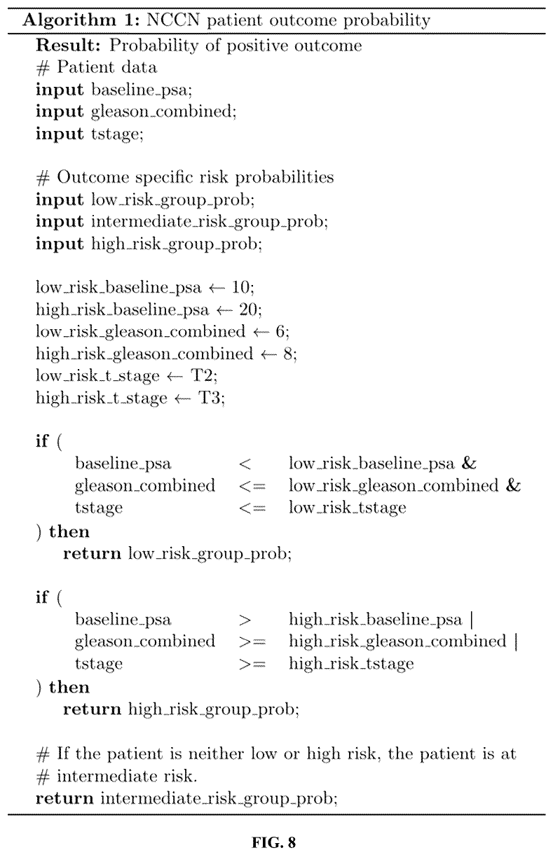

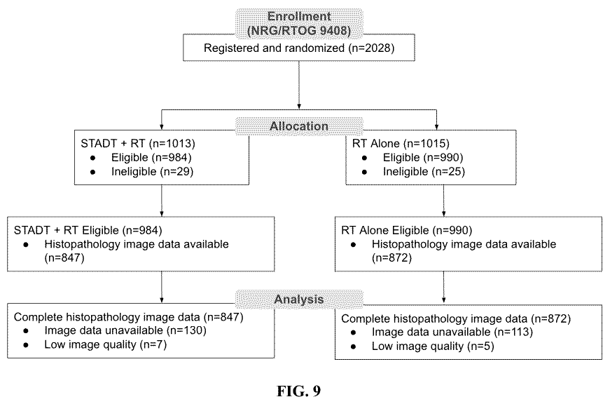

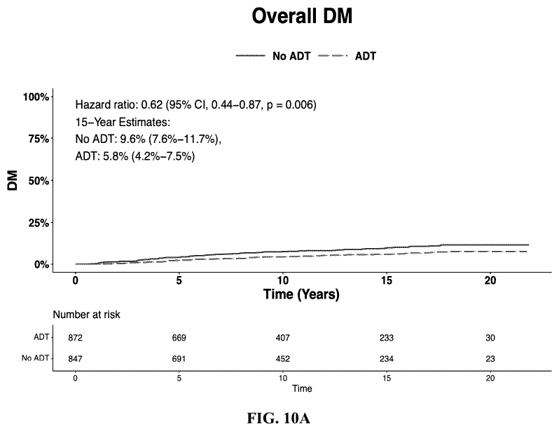

The NCCN model served as the baseline comparator, as shown in FIG. 8. Three variables—Gleason score, tumor t-stage, and baseline PSA—were used to group patients into low-, intermediate-, and high-risk groups.

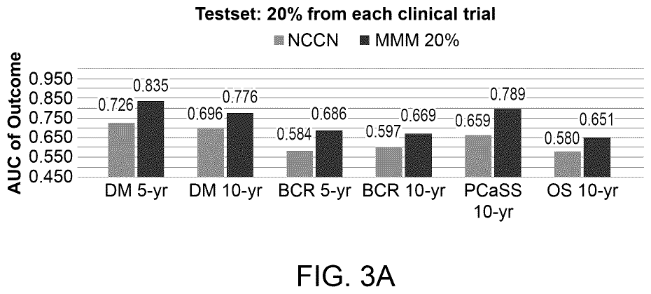

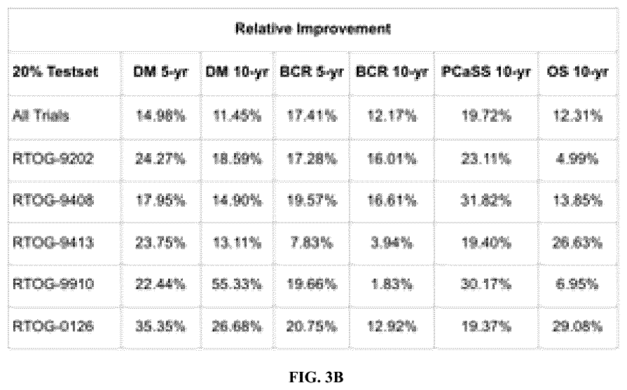

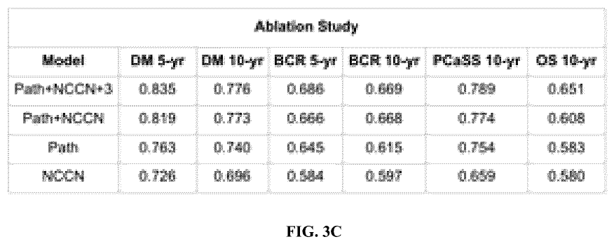

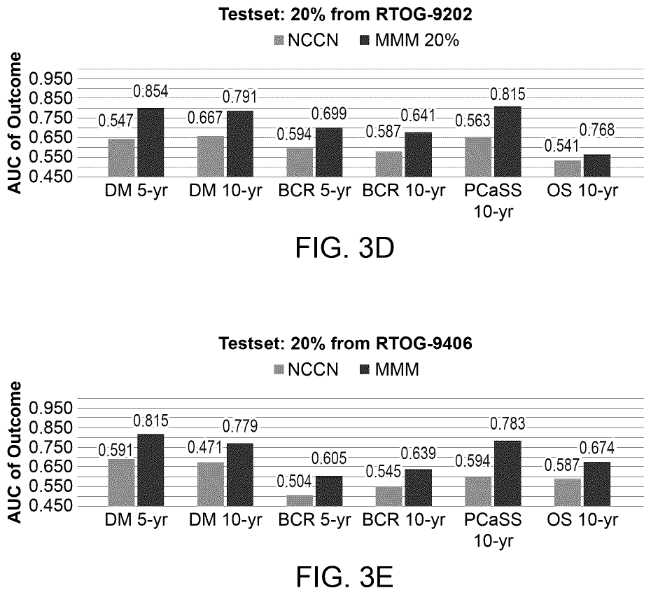

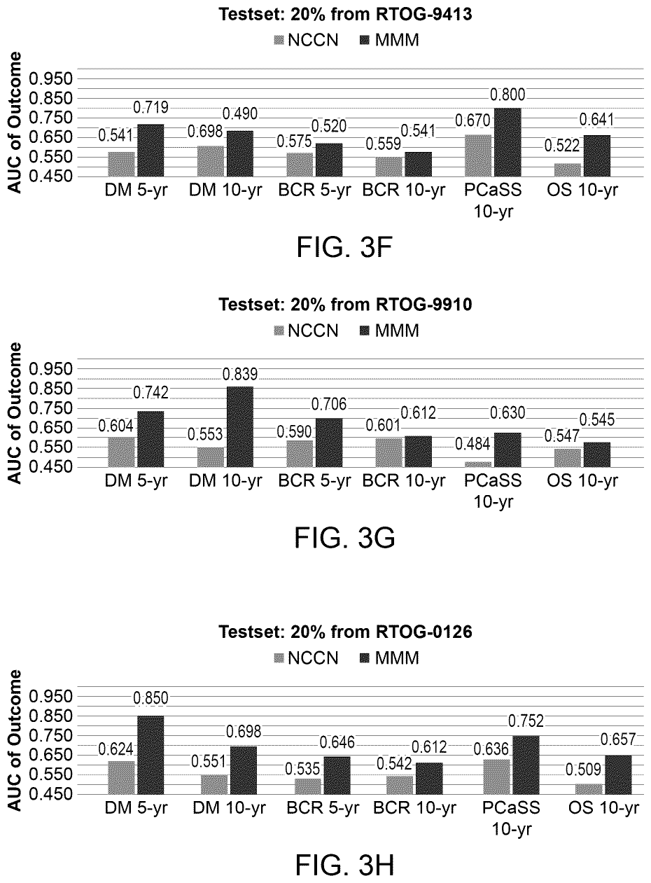

The results are shown in FIGS. 3A-H. A separate model was trained for each outcome and time point. In FIG. 3A and FIG. 2D-H, the blue bars represent the performance of an MMAI model trained on a specific task and the gray bars represent the performance of the corresponding NCCN model. FIG. 2B shows the relative improvement of the MMAI over NCCN across the outcomes and across the subsets of the test set that come from the five trials. The MMAI model consistently outperformed the NCCN model across all tested outcomes. The relative improvement in AUC varied from 11.45% up to 19.72%. Further, the trial subsets unanimously saw a relative improvement over NCCN.

To evaluate the effects of the various data components specific to the MMAI model, an ablation study was run. Additional MMAI models were trained using the following data setups: NCCN variables only, pathology images only, pathology images+the NCCN variables (combined Gleason score, t-stage, baseline PSA), and pathology images+NCCN variables+3 additional variables used in the model (age, Gleason primary, Gleason secondary). Each additional data component improved performance, with the full setup (pathology, 6 clinical variables) yielding the best results (FIG. 2C).

The MMAI system substantially outperformed the NCCN risk stratification tool, encoded as a model, at predicting four key future outcomes for patients: distant metastasis, biochemical recurrence, prostate cancer-specific survival, and overall survival. By creating a deep learning architecture that simultaneously ingested multiple data types (of variable sizes) from a patient, as well as clinical data, a deep learning system capable of inferring long-term patient outcomes with substantially higher accuracy than established clinical models was built.

Methods and systems as described herein may leverage robust and large-scale clinical data from five different prospective, randomized, multinational trials with 10-20 years of patient follow-up for 5,654 patients across a varied population. Validation of these prognostic classifiers on a large amount of clinical trials data—in the intended use population—uniquely positions these tools as aids to therapeutic decision-making. Critical flaws of similar, genomics-based assays are their high costs and lengthy turnaround times; AI tools do not share these limitations, which substantially lower the barriers to large-scale international adoption. Nearly 60% of the world has access to the internet, yet only about 4% (the US population) have easy access to genomics-based assays. The growing adoption of digital histopathology, coupled with internet connectivity, may support global distribution of AI-based prognostic and predictive testing, enabling low-cost access to critical therapy personalization.

### Methods

Tabular Pipeline. The tabular clinical data was separated into numerical and categorical variables. Numerical variables were whitened (mean subtraction+max normalization) to the range [−1,1]. Categorical variables were treated as one-hot vectors that are embedded into 2-3 dimensional vectors following conventional word-to-vec techniques, with a dimensionality given by the formula D=Round(1.6·num_categories^{0.56}). A TabNet model that takes in a concatenation of categorical and numerical variables as input was used (parameters: learning rate 0.2, Adam optimizer with step learning rate scheduler, batch size of 1024, 50 max epochs with early stopping patience of 10 epochs).

Image Pipeline. A ResNet50 model, together with the MoCo-v2 training protocol (parameters: learning rate=0.03 with a cosine learning rate schedule, moco-t=0.2, multilayer perceptron head, batch size of 256, MoCo-v2 data augmentation, 200 epochs) was used to train the SSL models used in the system architecture of FIG. 2B. For each held-out test set in FIG. 3A, only the images of the training data were used to pre-train a corresponding SSL model. Certain image patches were oversampled using nucleic density sampling as described elsewhere herein. Once SSL pre-training was complete, all W×H patches were fed into the SSL pretrained ResNet50 model to generate a W×H×128 feature quilt for each image quilt. The final image model used for prediction was a 2-layer CNN model with batchnorm and dropout, which takes in the feature tensors as input. The final CNN model was trained with batch size of 32, 150 maximum epoch and Adam optimizer with learning rate of 0.01 and step learning rate scheduler.

Downstream Pipeline. A joint fusion approach was used to leverage information from both modalities (image and tabular features). The images were featurized to feature tensors and fed into the final image model to produce a feature vector, while the tabular features were separately fed into the TabNet model to produce another feature vector. Two fully-connected layers processed the concatenated feature vectors of each pipeline and output prediction probabilities. For patients with missing histopathology data, the image-based feature vector was zeroed prior to concatenation.

Dataset Preparation. Full patient-level baseline clinical data, digitized histopathology slides of prostate biopsies, and longitudinal outcomes from five landmark, large-scale, prospective, randomized, international clinical trials containing 5,654 patients, 16,204 histopathology slides, and 10-20 years of median patient follow-up was used. These trials were RTOG 9202, 9408, 9910, 0126, and 9413 (FIG. 2C). These trials randomized across various combinations of radiotherapy (RT) and androgen-deprivation therapy (ADT): RT+short-term ADT (RTS), RT+medium-term ADT (RTM), RT+long-term ADT (RTL), and dose and volume levels of RT (RT+). The slides were digitized over a period of two years by NRG Oncology using a Leica Biosystems Aperio AT2 digital pathology scanner at a resolution of 20×. The histopathology images were manually reviewed for quality and clarity. Digital slides were converted into a single image quilt of size 200 by 200 patches for each unique patient prior to model training. Each clinical trial collected slightly different clinical variables. Six clinical variables that were available across all trials (combined Gleason, Gleason primary, Gleason secondary, t-stage, baseline PSA, age), along with the digital histopathology, were used for model training and validation.

Tissue segmentation. After slicing the slides into 256×256 pixel patches at 10× zoom, developed an artifact classifier was developed by training a ResNet-18 to classify whether a patch showed usable tissue, or whether it showed whitespace or artifacts. The artifact classifier was trained for 25 epochs, optimized using SGD with a learning rate of 0.001. The learning rate was reduced by 10% every 7 epochs. 3661 patches (tissue vs not tissue) were manually annotated, and the classifier was trained on 3366 of them, achieving a validation accuracy of 97.6% on the remaining 295. This artifact classifier was then used to segment tissue sections during image quilt formation.

Nucleic Density Sampling. Due to significant variation in stain intensity and stain degradation, readily-available pretrained models for nuclei detection and segmentation were unable to accurately detect nuclei in a majority of our slides. To overcome this, a nuclei detector was trained using the YOLOv5 (github.com/ultralytics/yolov5) object detection method.

In order to train the YOLOv5 model, a representative sample of 34 handpicked slides were manually labeled using the QuPath image analysis platform. First, the “Simple tissue detection” module was used to segment tissue. Next, the “Watershed cell detection” module was used to segment cells, with manually tuned parameters for each slide. A YOLOv5-Large model was then trained on the annotations from 29 of the slides and evaluated on the remaining 5. This model was trained using 256×256 patches at 10× zoom.

Model Performance Metrics. (AUC) For each model and each outcome, the time-dependent receiver operating characteristic, accounting for competing events, was estimated using the R-package timeROC. This is a curve of time-dependent sensitivities and specificities computed by sweeping a threshold t in the interval [0,1] and defining a model's prediction as ŷ=P>t, where P is the outcome probability outputted by the model. The area under this curve defines the model's performance on the task at hand.

NCCN Model. The NCCN model was coded according to the algorithm in FIG. 8, using three clinical variables—Gleason, t-stage, and baseline PSA—to bin patients into low, medium, and high-risk groups.

### Example 2—A Predictive AI Biomarker for Prostate Cancer Treatment

A study was performed to determine a predictive biomarker for response to androgen deprivation therapy (ADT) in addition to radiotherapy. In this study, five international phase III randomized trials with long term follow-up were acquired, digitized, trained, and validated to predict which men with localized prostate cancer derive greater benefit from the addition of ADT to radiotherapy.

### Methods

Patients and Trials. A pre-specified analysis plan was approved through NRG Oncology, a National Clinical Trials Network (NCTN) group funded by the National Cancer Institute (NCI). Trials were included if they were randomized phase III trials, had >8 years of median follow-up, were conducted in men with localized prostate cancer, were treated with radiotherapy with or without ADT, and had stored histopathology slides in the NRG biobank. Trials testing the use of chemotherapy were excluded. This identified five phase III randomized trials (NRG/RTOG 9202, 9408, 9413, 9910, and 0126) [7-11].

Details of each trial can be found in Table 1. Briefly, NRG/RTOG 9202 enrolled men with intermediate- and high-risk prostate cancer and randomized patients to radiotherapy with 4- vs. 24-months of ADT. NRG/RTOG 9408 enrolled men with low-, intermediate-, and high-risk prostate cancer and randomized men to radiotherapy with or without 4-months of ADT. NRG/RTOG 9413 enrolled men with intermediate- and high-risk prostate cancer and was a 2×2 factorial trial with randomizations to 4-months of ADT sequencing and use of pelvic nodal radiotherapy. NRG/RTOG 9910 enrolled men with intermediate-risk prostate cancer, and randomized patients to radiotherapy with 4- vs 9-months of ADT. NRG/RTOG 0126 randomized intermediate-risk patients to lower vs higher doses of radiotherapy alone. Trials that included the use of ADT consisted of combined androgen blockade with an LHRH agonist and an anti-androgen. Patients with missing data (clinical or available histopathology slides) were excluded from all analyses.

TABLE 1
Study Summary and patient baseline characteristics for all NRG/RTOG trials used.

| | RTOG- | RTOG- | RTOG- | RTOG- | RTOG- |
|---|---|---|---|---|---|
| Total | 9202 | 9413 | 9910 | 0126 | 9408 |
| (N = | (N = | (N = | (N = | (N = | (N = |
| 5688) | 1240) | 830) | 974) | 1050) | 1594) |

Assigned treatment arm

| | | | | | | |
|---|---|---|---|---|---|---|
| Hormones | 627 | 627 | — | — | — | — |
| 2 + y post RDT | (11%) | (51%) | | | | |
| Hormones | 613 | 613 | — | — | — | — |
| until end of | (11%) | (49%) | | | | |
| RT | | | | | | |
| Neoadj. | 205 | — | 205 | — | — | — |
| ST-ADT + | (3.6%) | | (25%) | | | |
| PORT | | | | | | |
| Neoadj. | 210 | — | 210 | — | — | — |
| ST-ADT + | (3.7%) | | (25%) | | | |
| WPRT + | | | | | | |
| Boost | | | | | | |
| PORT + | 196 | — | 196 | — | — | — |
| Adj. ST-ADT | (3.4%) | | (24%) | | | |
| WPFT + | 219 | — | 219 | — | — | — |
| Boost + Adj. | (3.9%) | | (26%) | | | |
| ST-ADT | | | | | | |
| 3D/IMRT | 521 | — | — | — | 521 | — |
| 70.2 | (9.2%) | | | | (50%) | |
| 3D/IMRT | 529 | — | — | — | 529 | — |
| 79.2 | (9.3%) | | | | (50%) | |
| RT alone | 806 | — | — | — | — | 806 |
| | (14%) | | | | | (51%) |
| RT + 18 | 481 | — | — | 481 | — | — |
| Wks of HT | (8.5%) | | | (49%) | | |
| RT + 26 | 788 | — | — | — | — | 788 |
| Wks of HT | (14%) | | | | | (49%) |
| RT+ 36 | 493 | — | — | 493 | — | — |
| Wks of HT | (8.7%) | | | (51%) | | |

Age

| | | | | | | |
|---|---|---|---|---|---|---|
| Median | 71 | 70 (66- | 70 (65- | 71 (656- | 71 (65- | 71 (66- |
| (IQR) | (66-74) | 74) | 74) | 75) | 74) | 74) |

Race

| | | | | | | |
|---|---|---|---|---|---|---|
| African | 942 | 153 | 209 | 166 | 108 | 306 |
| American | (17%) | (12%) | (25%) | (17%) | (10%) | (19%) |
| White | 4524 | 1054 | 574 | 767 | 909 | 1220 |
| | (80%) | (85%) | (69%) | (79%) | (87%) | (77%) |
| Other | 192 | 27 (2.2%) | 41 (4.9%) | 40 (4.1%) | 19 (1.8%) | 65 (4.1%) |
| | (3.4%) | | | | | |
| Missing | 1 | — | — | 1 | — | — |

Baseline PSA (ng/ml)

| | | | | | | |
|---|---|---|---|---|---|---|
| Median (IQR) | 11 (7- | 20 (11-40) | 23 (13-35) | 11 (6.8-15) | 7.6 (5.4- | 8.1 (5.8- |
| | 18) | | | | 10) | 12) |
| Missing | 10 | 0 (0%) | 0 (0%) | 0 (0%) | 0 (0%) | 0 (0%) |
| | (0.2%) | | | | | |
| <4 | 333 | 38 (3.1%) | 3 (0.4%) | 45 (4.7%) | 102 (9.7%) | 145 |
| | (5.9%) | | | | | (9.1%) |
| 4-10 | 2247 | 223 (18%) | 130 (16%) | 379 (39%) | 640 (61%) | 874 (55%) |
| | (40%) | | | | | |
| 10-20 | 1833 | 353 (28%) | 204 (25%) | 448 (46%) | 308 (29%) | 570 (36%) |
| | (33%) | | | | | |
| 20+ | 1215 | 625 (50%) | 493 (59%) | 92 (9.5%) | 0 (0%) | 5 (0.3%) |
| | (20%) | | | | | |

Tumor Stage

| | | | | | | |
|---|---|---|---|---|---|---|
| T1 | 1973 | — | 130 (16%) | 493 (51%) | 575 (55%) | 775 (49%) |
| | (35%) | | | | | |
| T2 | 2679 | 561 (47%) | 400 (49%) | 424 (44%) | 475 (45%) | 819 (51%) |
| | (48%) | | | | | |
| T3-T4 | 982 | 638 (53%) | 287 (35%) | 57 (5.9%) | — | — |
| | (17%) | | | | | |
| Missing | 54 | 41 | 13 | — | — | — |

Nodal Stage

| | | | | | | |
|---|---|---|---|---|---|---|
| N0 | 1301 | 126 (10%) | 44 (5.3%) | 72 (7.7%) | 992 (94%) | 67 (4.2%) |
| | (23%) | | | | | |
| N1-3 | 63 | 53 (4.3%) | 9 (1.1%) | 1 (0.1%) | — | — |
| | (1.1%) | | | | | |
| NX | 4281 | 1061 | 777 (94%) | 858 (92%) | 58 (5.5%) | 1527 |
| | (76%) | (86%) | | | | (96%) |
| Missing | 119 | 82 | — | — | — | 37 |

Gleason

| | | | | | | |
|---|---|---|---|---|---|---|
| <7 | 2082 | 405 (34%) | 196 (28%) | 271 (28%) | 162 (15%) | 1048 |
| | (37%) | | | | | (61%) |
| 7 | 2694 | 398 (34%) | 365 (44%) | 595 (61%) | 899 (86%) | 437 (28%) |
| | (48%) | | | | | |
| 8-10 | 788 | 293 (25%) | 235 (28%) | 109 (11%) | — | 151 |
| | (14%) | | | | | (9.7%) |
| Missing | 119 | 82 | — | — | — | 37 |

Risk Group

| | | | | | | |
|---|---|---|---|---|---|---|
| Low | 534 | — | — | 7 (1%) | — | 577 (34%) |
| | (9.5%) | | | | | |
| Intermediate | 3001 | 232 (19%) | 130 (18%) | 711 (74%) | 1050 | 878 (56%) |
| | (53%) | | | | (100%) | |
| High | 2091 | 991 (81%) | 700 (84%) | 249 (26%) | — | 151 |
| | (37%) | | | | | (9.7%) |
| Missing | 62 | 17 | — | 8 | — | 37 |

Legend: 3D/IMRT=intensity-modulated radiation therapy; Wks=weeks; RT=radiation therapy; HT=hormone therapy: IQR=interquartile range; PSA=prostate-specific antigen; ng/ml=nanograms per milliliter.

In some aspects, the example describes the development and validation of a predictive biomarker that can identify differential benefit from the addition of ADT to radiotherapy in localized prostate cancer. One endpoint to define benefit was time to distant metastasis, measured from time of randomization until development of distant metastasis or last follow-up. Additional endpoints included prostate cancer-specific mortality (PCSM), metastasis-free survival (MFS), and overall survival (OS).

Histopathology pipeline. Pre-treatment biopsy slides from the NRG Oncology Biospecimen Bank were independently digitized without access to clinical outcomes data. The slides were digitized using a Leica Biosystems Aperio AT2 digital pathology scanner at a resolution of 20×. The histopathology images were manually reviewed for quality and clarity.

The digitized histopathology images were sent to a team of AI scientists for feature extraction and were blinded to clinical data at this point. For each unique patient, the tissue across all of their digital slides were cropped out and pasted into a single image quilt of size 51,200×51,200 pixels. Image quilts were divided into 256×256 pixel patches, which yielded 200×200 patches per image quilt. A YOLOv5 object detection model was trained to identify nuclei in histopathology images to compute the number of nuclei per patch for downstream training.

A Resnet-50 object classification model was trained on these image patches using self-supervised learning (SSL) [16]. The MoCo-v2 training protocol was employed without access to any clinical or outcomes data [17]. Over 6 million tissue patches across the training dataset were fed through the model >100 times. Image patches with higher nuclei counts were oversampled in order to learn salient histopathology features for downstream prediction.

Model development and validation. The training cohort for model development included patients from NRG/RTOG 9910 and 0126. Since NRM/RTOG 9910 and each contribute to one treatment type of interest (RT+ST-ADT vs. RT only, respectively), inverse probability of treatment weighting (IPTW) was used to ensure that patients in two treatment types had comparable selected baseline clinical characteristics, including age, baseline PSA, T-stage, Gleason score and primary/secondary pattern. Then, the model development cohort was further stratified by treatment type and randomly split into training (60%) and tuning (40%) sets for model training and hyperparameter tuning, respectively [18, 19]. Clinical data, image data, and treatment types (rx) were used as inputs to a multi-modal predictive model architecture (schematically illustrated in FIG. 2C).

Clinical Data Preprocessing. Categorical clinical variables (T-stage, Gleason score and primary/secondary Gleason pattern) and binary treatment type (0 for radiotherapy [RT] alone, 1 for RT with ST-ADT) were fed through neural network embedding layers to generate continuous vector embeddings. Groupings are as follows: Gleason total (≤6, 7, 8, and ≥9), both primary and secondary Gleason patterns (≤3, 4, and 5), T-stage (Tx, T0, T1a, T1b, T1c or T1, T2a, T2b, T2c or T2, T3a, T3b, T3c or T3, T4a, T4b, T4). Continuous clinical variables (age, baseline PSA) were standardized based on the mean and standard deviation of the training data.

Image Feature Extraction Model Development. For each patch from a patient's histopathology images, a 128-dimensional feature vector was extracted using the self-supervised pre-trained Resnet-50 image feature extraction model and was standardized based on the mean and standard deviation of the training data. All the patch-level feature vectors from the same patient were stacked to form an image feature tensor, which was fed to the downstream predictive model.

Inverse Probability Treatment Weighting. As the development set comprised two phase III randomized trials (NRG/RTOG 9910 and 0126), inverse probability of treatment weighting (IPTW) was used to ensure that patients in two treatment types had comparable clinical baseline characteristics. Propensity score was calculated using a logistic regression model with elastic net penalty, where treatment types were regressed against patients' age, baseline PSA, Gleason score, Gleason primary/secondary patterns, and T-stage variables. To mitigate the high variability introduced by large weights, IPTW weights were trimmed based on the 1st and 99th percentiles.

### Downstream Predictive Model Development

The downstream predictive model took the image feature tensor, preprocessed clinical data, and treatment type (rx) as input for each patient (FIG. 2C). An attention multiple instance learning network was employed to learn a weight for each patch from the patient. A single 128-dimensional image vector was generated from the image feature tensor for each patient by taking the weighted sum of the image vectors of all patches from the same patient, where the weights were learned by the attention mechanism. A concatenation of this single 128-dimensional image vector, preprocessed clinical data, and treatment type was further processed through the joint fusion pipeline to effectively learn predictive feature encodings of differential treatment benefit from the addition of ST-ADT to radiotherapy.

The multi-modal predictive model was trained in a multitask manner. The first task was to predict the relative risk of DM using the factual rx (“Task 1” in FIG. 2C). The image, clinical, and factual rx vectors were concatenated and fed through a few layers of fully connected neural networks to produce a continuous score for each patient that estimates the relative risk of DM (sometimes referred to as “factual model prediction score” herein). The negative log-partial likelihood was used as the training objective for the first task and the factual model prediction scores were the estimated relative log hazards.

The negative log-partial likelihood loss was parameterized by the model weights θ and formulated as follows:

$$loss ( θ ) := - \frac{1}{{N}_{E = 1}} {\sum }_{i : {E}_{i} = 1} ( {f}_{θ} ( {x}_{i} ) - log {\sum }_{j ∈ R ( {T}_{i} )} {e}^{{f}_{θ} ( {x}_{j} ) )}$$

where the values Tᵢ, Eᵢ, and xᵢ are the event time or time of last follow-up, an indicator variable for whether the event is observed, and the model input for the ith observation, respectively. The function ƒ_{θ} represents the factual branch of the multi-modal model, and ƒ_{θ}(x) is the estimated relative risk given an input x. The value N_{E=1} represents the number of patients with an observable event. The set of patients with an observable event is represented as Eᵢ=1. The risk set [symbol] (t)={i: Tᵢ≥t} is the set of patients still at risk of failure at time t. Breslow's approximation was used for handling tied event times.

Based on the estimated relative risk on the first task, the second task was to predict the delta score, defined as the difference in factual model prediction score and counterfactual model prediction score (“Task 2” in FIG. 2C). To this end, a counterfactual rx variable was created by toggling the patient's factual rx (RT for patients who received RT with short-term ADT, and vice versa). The counterfactual rx variable was fed through the same rx embedding layer and concatenated with the image and clinical vectors. Then, the concatenated vectors were fed through the same fully connected neural network layers yielding another continuous score (sometimes referred to as “counterfactual model prediction score” herein). For patients who received RT alone, delta would be the factual model prediction score minus the counterfactual prediction score; whereas for patients with RT+ST-ADT treatment, delta would be the counterfactual prediction score minus the factual model prediction score. The delta indicates the magnitude of therapeutic benefit for each patient, where a larger delta suggests a larger benefit from additional ST-ADT, and vice versa.

For this predictive task, the delta loss was designed and used as the training objective. The delta loss characterized the deviation between the predicted delta scores and the expected delta scores. The expected delta scores were dependent on patients falling into one of four subgroups based on their treatment types and DM outcomes as follows: (a) Subgroup A of metastasis-free patients who received RT alone; (b) Subgroup B of patients who received RT alone and had DM; (c) Subgroup C of metastasis-free patients who received RT with ST-ADT; (d) Subgroup D of patients who received RT with ST-ADT and had DM. For Subgroup A, the delta was expected to be close to 0 as the patients had no DM when receiving RT alone treatment and additional ST-ADT would not affect their risk of DM; for Subgroup B, the delta was expected to be greater than or equal to 0 since the patients might benefit from additional ST-ADT treatment; for Subgroup C, the delta was expected to be greater than 0 since the patients were free of DM when receiving additional ST-ADT; finally, Subgroup D's delta was expected to be close to 0 as the patients had DM even if they received additional ST-ADT treatment. During training, the model was penalized when the delta scores did not fall in the expected range described above. The training objective for the predictive task was defined using the softplus function.

During training, the weighted sum of both losses from the prognostic task and the predictive task were approximated, and each data point was weighted by its IPTW weight. Once the model was trained, a cutoff was selected at the 67th percentile of the delta scores in the development set such that all patients in the validation set with a delta score greater than the cutoff were considered to be biomarker positive, with predicted benefit of additional ST-ADT, and those with a delta score less than the cutoff were considered to be biomarker negative. The final model was chosen based on the lowest ratio of IPTW-weighted hazard ratios of biomarker positive and negative subgroups on the tuning set.

The multi-modal predictive model optimized the difference in the magnitude of ADT benefit outputting the continuous score delta described herein above. The 67th percentile of the delta scores in the development set was selected as the cutoff threshold as it maximized the difference between biomarker subgroup treatment effects in the tune set and would result in reasonably sized biomarker subgroups for clinical utility. Patients with a delta score greater than the cutoff are identified as biomarker positive and vice versa (FIG. 9). After the biomarker was locked, it was provided to independent biostatisticians to perform clinical validation of the biomarker in NRG/RTOG 9408.

### Statistical Analysis

The validation cohort characteristics by biomarker status were reported and compared using chi-square test or Fisher's exact test in the presence of low cell counts for categorical variables, and Wilcoxon rank-sum test for continuous variables. Time to DM and PCSM were analyzed using the cumulative incidence function, with death without the corresponding event as a competing risk. Outcomes for patients treated with and without ST-ADT were compared using the Gray's test [20]. Fine and Gray regression was also performed to estimate the subdistribution hazard ratio (sHR) and 95% confidence interval (CI) for the ST-ADT treatment effect [21]. A test for biomarker-treatment interaction was performed to evaluate this predictive biomarker. Treatment effects of the biomarker positive and negative subgroups were similarly assessed as the overall validation cohort to measure the relative treatment effect between arms. Fifteen-year restricted mean survival times were reported to provide alternative estimates given non-proportional hazards were observed [10].

Exploratory subgroup analyses were performed based on NCCN risk stratification, and the primary analysis was reanalyzed within NCCN low- and intermediate-risk patients. Statistical analyses were performed using R, version 3.5.1 (R Foundation for Statistical Computing, Vienna, Austria). All statistical tests were two-sided and p-values less than 0.05 were deemed statistically significant.

### Results

Patient Characteristics. Of the 7,752 eligible patients enrolled on the five phase III randomized trials, 5,825 (75.1%) patients had available pre-treatment biopsy tissue at the NRG Biospecimen Bank. Of these patients, 5,727 (98.3%) were eligible for inclusion with high quality digital pathology data. Of these patients, 39 patients with TURP samples were further excluded from the validation cohort (NRG/RTOG 9048).

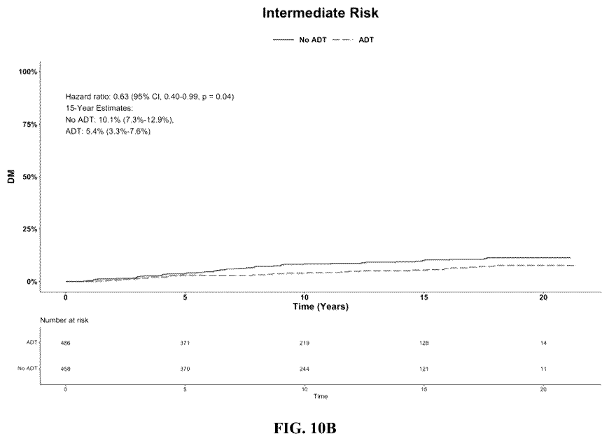

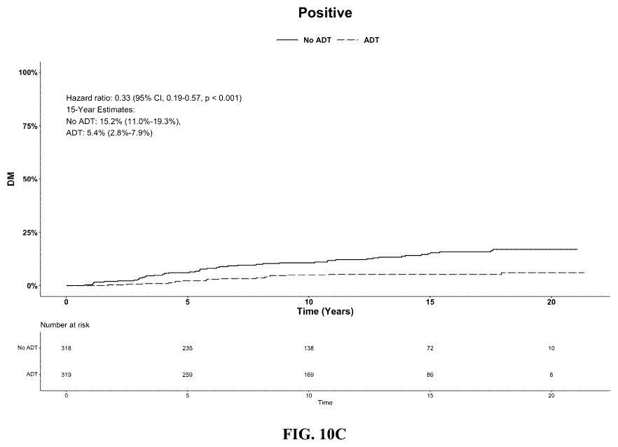

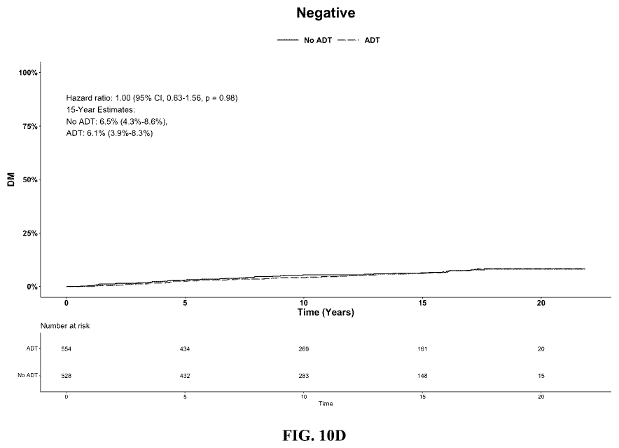

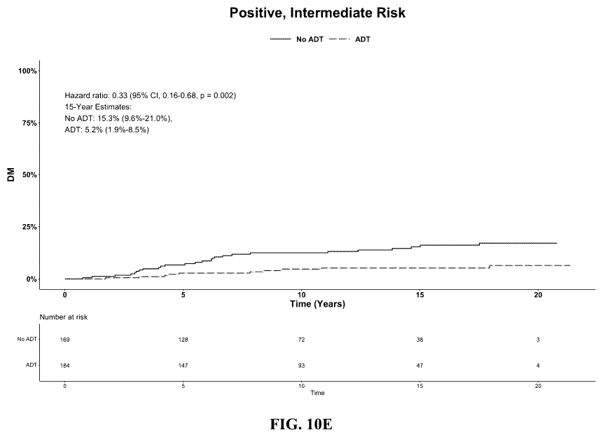

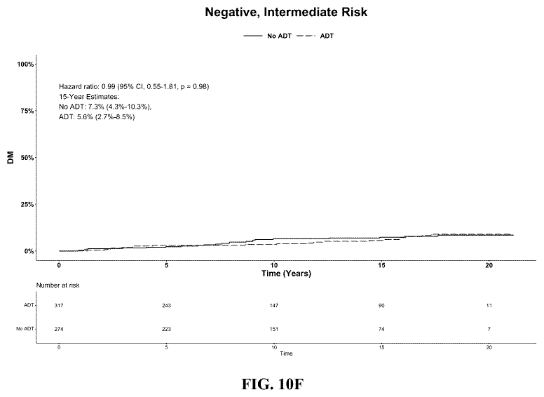

The development cohort for the downstream predictive model had 2,024 patients with a median follow-up of 10.6 years, and 1,050 (52%) patients received radiotherapy alone and 974 (48%) patients received RT with ST-ADT (Table 2). The median PSA was 9 ng/ml (interquartile range [IQR], 6-13), 87% had intermediate-risk disease, and the median age was 71 years (IQR, 65-74). The validation cohort (NRG/RTOG 9408) comprised 1,594 patients with a median follow-up of 14.9 years, with balanced treatment types (RT alone=806 patients, and RT plus ST-ADT=788 patients; FIG. 10 and Table 3). The median PSA was 8 ng/mL (IQR, 6-12), 56% had intermediate-risk disease, and the median age was 71 years (IQR, 66-74). There were no significant differences in the baseline characteristics between treatment types, and the evaluable cohort of NRG/RTOG 9408 was representative of the published overall trial cohort (Table 3).

TABLE 2
Patient baseline characteristics of the development cohort

| | | Before Reweighting | | | |
|---|---|---|---|---|---|
| | | RT + ST- | | After Reweighting | |
| | Overall | ADT | RT | RT + ST- | |
| Characteristic | N = 2024 | N = 974 | N = 1050 | ADT | RF |

Age

| | | | | | |
|---|---|---|---|---|---|
| Median | 71 (65-74) | 71 (65-75) | 71 (65-74) | 71 (66-75) | 70 (64-74) |
| (IQR) | | | | | |

Baseline PSA (mg/mL)

| | | | | | |
|---|---|---|---|---|---|
| Median | 9 (6-13) | 11 (7-15) | 8 (5-10) | 8 (6-13) | 8 (6-12) |
| (IQR) | | | | | |
| Missing | 10 | 10 | 0 | 189 | 0 |

Tumor Stage

| | | | | | |
|---|---|---|---|---|---|
| T1 | 53% | 51% | 55% | 53% | 53% |
| T2 | 44% | 44% | 45% | 43% | 47% |
| T3-T4 | 2.8% | 5.9% | 0% | 4.1% | 0% |

Gleason Score

| | | | | | |
|---|---|---|---|---|---|
| <7 | 21% | 28% | 14% | 21% | 20% |
| 7 | 74% | 61% | 86% | 67% | 80% |
| 8-10 | 5.4% | 11% | 0% | 12% | 0% |

Risk Group

| | | | | | |
|---|---|---|---|---|---|
| High | 12% | 26% | 0% | 21% | 0% |
| Intermediate | 87% | 74% | 100% | 78% | 100% |
| Low | 0.3%% | 0.36% | 0% | 1.0% | 0% |
| Missing | 8 | 8 | 0 | 166 | 0 |

Legend: RT = radiation therapy; ADT = androgen-deprivation therapy; IQR = interquartile range; PSA = prostate-specific antigen; ng/mL = nanograms per milliliter.

TABLE 3
Patient baseline characteristics for NRG/RTOG 9408 histopathology-imaged patients.

| | RTOG 9408 Full Cohort | | | | RTOG 9408 Imaged Cohort | | |
|---|---|---|---|---|---|---|---|
| | N = 1974 | | | | N = 1594 | | |
| | Overall | Imaged | Not | | ADT | ADT | |
| | (N = | (N = | Available | | (N = | (N = | P- |
| characteristic | 1974) | 1594) | (N = 380) | P-value | 788) | 806) | value |
| Age | | | | >0.9 | | | 0.14 |
| Median (IQR) | 71 (66- | 71 (66- | 70 (66-74) | | 70 (66- | 71 (66- | |
| | 74) | 74) | | | 74) | 74) | |
| Missing | 1 | 0) | 1 | | — | — | |
| Race | | | | 0.3 | | | 0.8 |
| African | 394 | 306 | 88 (23%) | | 156 | 150 | |
| American | (20%) | (19%) | | | (20%) | (19%) | |
| White | 1497 | 1220 | 277 (73%) | | 596 | 624 | |
| | (76%) | (77%) | | | (76%) | (77%) | |
| Other | 80 | 65 | 15 (3.9%) | | 34 | 31 | |
| | (4.1%) | (4.1%) | | | (4.3%) | (3.8%) | |
| Missing | 3 (0.2%) | 3 (0.2%) | 0 (0%) | | 2 | 1 (0.1%) | |
| | | | | | (0.3%) | | |
| KPS | | | | 0.7 | | | 0.5 |
| 70-80 | 154 | 126 | 28 (7.4%) | | 66 | 60 | |
| | (7.8%) | (7.9%) | | | (8.4%) | (7.4%) | |
| 90-100 | 1819 | 1468 | 351 (93%) | | 722 | 746 | |
| | (92%) | (92%) | | | (92%) | (93%) | |
| Missing | 1 | 0 | 1 | | — | — | |
| Baseline PSA (ng/ml) | | | | <0.001 | | | 0.3 |
| Median (IQR) | 8 (6-12) | 8 (6-12) | 7 (5-10) | | 8 (6-12) | 8 (6-12) | |
| Missing | 1 | 0 | 1 | | — | — | |
| <4 | 209 | 145 | 64 (17%) | | 79 | 66 | |
| | (11%) | (9.1%) | | | (10%) | (8.2%) | |
| 4-10 | 1089 | 874 | 215 (57%) | | 426 | 448 | |
| | (55%) | (55%) | | | (54%) | (56%) | |
| 10-20 | 669 | 570 | 99 (26%) | | 282 | 288 | |
| | (34%) | (36%) | | | (36%) | (36%) | |
| 20+ | 6 (0.3%) | 5 (0.3%) | 1 (0.3%) | | 1 | 4 (0.5%) | |
| | | | | | (0.1%) | | |
| Tumor Stage | | | | 0.8 | | | 0.2 |
| T1 | 962 | 775 | 187 (49%) | | 396 | 379 | |
| | (49%) | (49%) | | | (50%) | (47%) | |
| T2 | 1011 | 819 | 192 (51%) | | 392 | 427 | |
| | (51%) | (51%) | | | 50%) | (53%) | |
| Missing | 1 | 0 | 1 | | | | |
| Nodal Stage | | | | 0.5 | | | 0.8 |
| N0 | 80 | 67 | 13 (3.4%) | | 34 | 33 | |
| | (4.1%) | (4.2%) | | | (4.3%) | (4.1%) | |
| NX | 1893 | 1527 | 366 (97%) | | 754 | 773 | |
| | (96%) | (96%) | | | (96%) | (96%) | |
| Missing | 1 | 0 | 1 | | | | |
| Gleason Score | | | | 0.4 | | | 0.3 |
| <7 | 1212 | 969 | 243 (66%) | | 494 | 475 | |
| | (61%) | (62%) | | | (64%) | (61%) | |
| 7 | 535 | 473 | 98 (26%) | | 204 | 233 | |
| | (28%) | (28%) | | | (26%) | (30%) | |
| 8-10 | 180 | 151 | 29 (7.8%) | | 75 | 76 | |
| | (9.3%) | (9.7%) | | | (9.7%) | (9.7%) | |
| Missing | 47 | 37 | 10 | | 15 | 22 | |
| Risk Group | | | | 0.073 | | | 0.5 |
| Missing | 47 | 37 | 10 | | 15 | 22 | |
| Low | 676 | 528 | 148 (40%) | | 273 | 255 | |
| | (35%) | (34%) | | | (25%) | (33%) | |
| Intermediate | 1071 | 878 | 193 (52%) | | 425 | 453 | |
| | (56%) | (56%) | | | (55%) | (58%) | |
| High | 180 | 151 | 29 (7.8%) | | 75 | 76 | |
| | (9.3%) | (9.7%) | | | (9.7%) | (9.7%) | |

### ST-ADT Predictive Biomarker

The final locked model included 128 image features from the digital pathology slides, and 7 clinical variables, including age, combined Gleason score, primary and secondary Gleason, PSA, T-stage, and treatment type (RT with or without ST-ADT). The treatment type was used only for model development. Histopathology image features contributed to nearly 40% of model prediction (FIG. 11).

Applying the locked predictive model to the validation set, 543 patients (34%) were biomarker positive (predicted to benefit most from ST-ADT), and 1,051 patients (66%) were biomarker negative (predicted to derive little benefit from ST-ADT). Patients with biomarker positive disease were less likely to have Gleason Score 7 (24% vs 30%; p=0.02) when compared to patients with biomarker negative disease (Table 4).

TABLE 4
Patient baseline characteristics by
biomarker group in NRG/RTOG 9408.

| Character- | Overall | | Negative | | Positive | | |
|---|---|---|---|---|---|---|---|
| istic | N = 1594 | | N = 1051 | | N = 543 | | p-value |
| Age | | | | | | | 0.2 |
| Median | 71 | (66-74) | 71 | (66-74) | 70 | (66-74) | |
| (IQR) | | | | | | | |
| Race | | | | | | | 0.9 |
| African | 306 | (19%) | 198 | (19%) | 108 | (20%) | |
| American | | | | | | | |
| White | 1220 | (77%) | 810 | (77%) | 410 | (76%) | |
| Other | 65 | (4.1%) | 41 | (3.9%) | 24 | (4.4%) | |
| Unknown | 3 | (0.2%) | 2 | (0.2%) | 1 | (0.2%) | |
| Baseline PSA (ng/ml) | | | | | | | 0.8 |
| Median | 8 | (6-12) | 8 | (6-12) | 8 | (6-12) | |
| (IQR) | | | | | | | |
| <4 | 145 | (9.1%) | 101 | (9.6%) | 44 | (8.1%) | |
| 4-10 | 874 | (55%) | 576 | (55%) | 298 | (55%) | |
| 10-20 | 570 | (36%) | 372 | (35%) | 198 | (36%) | |
| >20 | 5 | (0.3%) | 2 | (0.2%) | 3 | (0.6%) | |
| Tumor Stage | | | | | | | 0.8 |
| T1 | 775 | (49%) | 509 | (48%) | 266 | (49%) | |
| T2 | 819 | (51%) | 542 | (52%) | 277 | (51%) | |
| Nodal Stage | | | | | | | 0.8 |
| N0 | 67 | (4.2%) | 45 | (4.3%) | 22 | (4.1%) | |
| Nx | 1527 | (96%) | 1006 | (96%) | 521 | (96%) | |
| Gleason Score | | | | | | | 0.020 |
| <7 | 969 | (62%) | 626 | (61%) | 3434 | (65%) | |
| 7 | 437 | (28%) | 311 | (30%) | 1126 | (24%) | |
| 8-10 | 151 | (9.7%) | 92 | (8.9%) | 59 | (11%) | |
| Missing | 37 | | 22 | | 15 | | |
| Risk Group | | | | | | | 0.2 |
| High | 151 | (9.7%) | 92 | (8.9%) | 59 | (11%) | |
| Intermediate | 878 | (56%) | 593 | (58%) | 285 | (54%) | |
| Low | 528 | (34%) | 344 | (33%) | 184 | (35%) | |
| Missing | 37 | | 22 | | 15 | | |

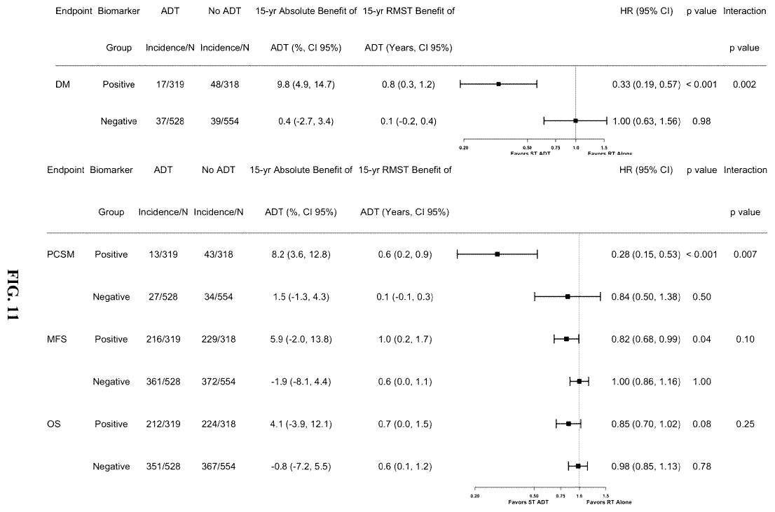

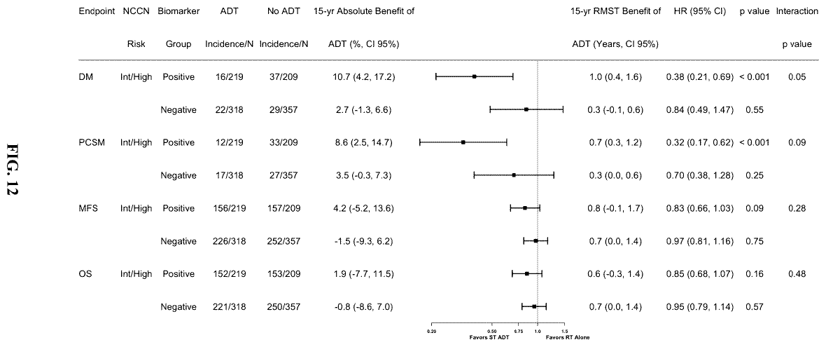

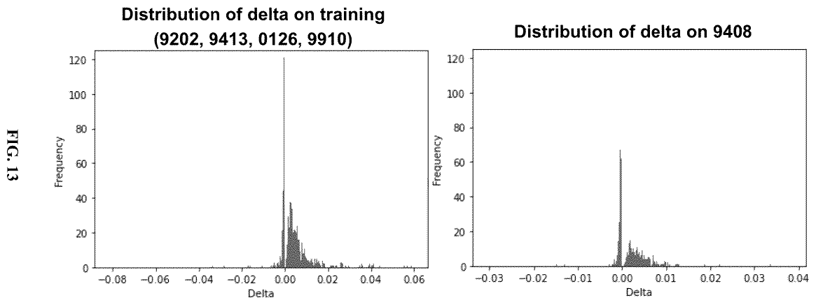

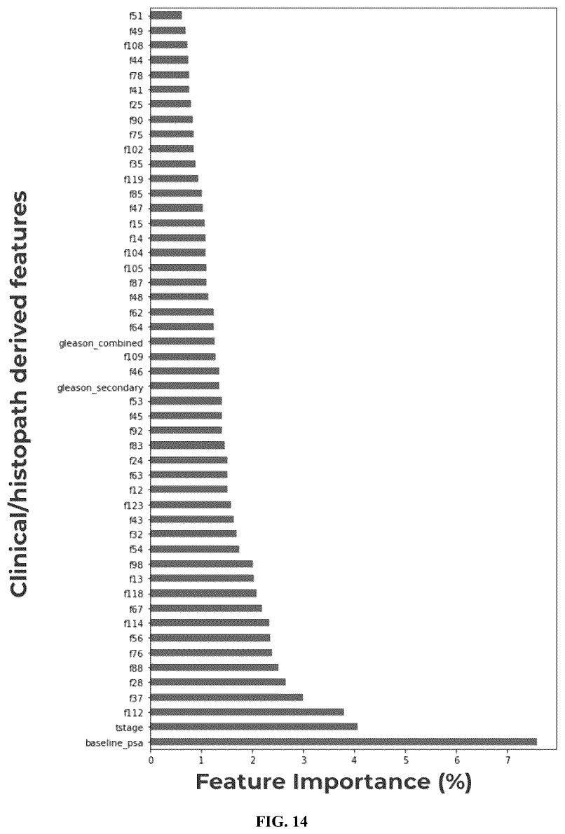

In the overall validation cohort, ST-ADT significantly prolonged the time to DM (sHR 0.64, 95% CI [0.45-0.90], p=0.01; FIG. 13). Applying the locked AI-derived biomarker to the validation set, the biomarker-treatment interaction was significant for time to DM (p-interaction=0.01; FIG. 14). In patients with biomarker positive disease, addition of ST-ADT significantly reduced the risk of DM compared to radiotherapy alone (sHR 0.34, 95% CI [0.19-0.63], p<0.001). In contrast, there was no significant difference between arms in the biomarker negative subgroup (sHR 0.92, 95% CI [0.59-1.43], p=0.71). The absolute benefit of ST-ADT, measured as the difference in DM risks between treatment arms at 15 years after randomization, was 10.5% (95% CI 5.4%-15.5%; FIG. 13) in biomarker positive patients. In contrast, in patients with biomarker negative disease there was a 0.5% (95% CI-2.8%-3.7%) reduction in 15-year DM risk from the addition of ADT.

On exploratory subset analysis, when restricting the analyses to solely patients with low- and intermediate-risk disease the results remained similar. Furthermore, similar biomarker-treatment interaction for DM in this subgroup of patients was observed (p-interaction=0.009; FIG. 15).

The secondary endpoint PCSM was also assessed (FIG. 14 and FIG. 15). In the overall validation cohort, ST-ADT significantly prolonged the time to event (sHR 0.52, 95% CI [0.35-0.78], p=0.001; FIG. 15). Patients receiving ST-ADT with biomarker positive disease had a significant improvement in PCSM (SHR 0.28, 95% CI 0.14-0.57, p<0.001) compared to radiotherapy alone. In contrast, despite the biomarker negative group being substantially larger with more events than the biomarker positive group, there were no significant improvements from additional ADT compared to RT alone in PCSM, sHR 0.74, 95% CI 0.45-1.22, p=0.24.

The use of AI in oncology may remain in its infancy with limited clinical deployment. Using novel deep learning methodology and leveraging image data from over 5,000 patients on five phase III randomized trials, we have successfully trained and validated a predictive biomarker to guide the use of ADT in men treated with radiotherapy in localized prostate cancer. These findings represent a long-sought milestone in prostate cancer and can be used to rapidly advance precision medicine for this common disease.

Across cancer types, histopathology information may be principally used for the purpose of diagnosis and tumor grading. In prostate cancer, Gleason grading may be modestly prognostic and has not been shown to function as a predictive biomarker for ADT use [22]. Thus, a plethora of tissue-based gene expression, serum, and imaging biomarkers have been created in hopes to fill this unmet need [12]. While some have demonstrated improvements in risk-stratification and prognostication [23], none have shown to function as predictive biomarkers with randomized trial validation.

As a patient's prognosis worsens (e.g., going from NCCN low- to high-risk), the recommendations to add ADT to radiotherapy strengthen. This is despite the evidence that NCCN risk groups are not predictive of ADT benefit [5]. To this point, this example demonstrates that biomarker positive and negative patients had similar baseline PSA, T-stage, and NCCN risk group distribution, with small differences in Gleason grade. This reinforces the notion that historical measures of tumor aggressiveness appear minimally correlated with which patients derive differential relative benefit from ADT.

Notwithstanding the limitations of Gleason grading, histopathology imagery may have significant value. There is a wealth of information within a histopathology image far beyond the current five grade groups. However, to capitalize on this information, the present example demonstrates that AI can be used to unlock this vast array of quantifiable data from digital pathology, without restricting the model solely to established human interpretable features.

Generally, model may encoutner challenges with the possibility of overfitting and failure to validate. Therefore, independent validation may be necessary to prove the performance of a biomarker. In the specific case of predictive biomarkers, which aim to provide information on which patients derive greater or lesser relative benefit, this may be performed within the context of a randomized trial of the treatment of interest to avoid confounding and bias between arms. Herein, NRG/RTOG 9408 was selected as it remains the largest published trial of radiotherapy with or without ST-ADT with very long-term follow-up. While there was clear benefit of ADT in unselected patients in this trial, the majority of patients enrolled had no demonstrable benefit. The results indicate that over 60% of the intermediate-risk patients enrolled on NRG/RTOG 9408 may avoid unnecessary treatment with ADT, and hence be spared the morbidity and costs associated with ADT.

The primary endpoint of time to DM was carefully selected to train the ST-ADT predictive biomarker. Other endpoints, such as biochemical recurrence, metastasis-free survival (MFS), and OS, all have clinical relevance, but in the context of localized prostate cancer biomarker development have notable limitations. ADT inhibits PSA production, and thus ADT is expected to delay biochemical recurrence irrespective of subgroup. Furthermore, the majority of biochemical recurrence events do not result in metastasis or death [22]. Therefore, it may be a less optimal endpoint for biomarker training to determine intrinsic tumor-specific benefit from ADT.

MFS and OS are important endpoints for determining the net effect of a given therapy and are the gold-standard for clinical trial design. However, they may be suboptimal endpoints for development of prostate cancer-specific biomarkers. This is because 78% of deaths in the validation cohort were not from prostate cancer, and only 12% of events in the MFS endpoint were from metastatic events. Thus, the strongest prediction models for MFS and OS may actually be driven by variables associated with death from non-prostate cancer causes (e.g., comorbid conditions). Importantly, despite the biomarker being trained for DM, it showed a clear differential impact of ADT by biomarker status for PCSM, a cancer driven endpoint.

As with any model, generalizability may be critical. Challenges may be raised in AI models derived from a limited number of centers and in cohorts with limited diversity. In contrast, NRG/RTOG enrolls patients from over 500 centers across primarily the USA and Canada from academic, community, and Veterans Affairs centers, and is well represented by racial minorities (20% of patients in the validation set were African American). This important real-world diversity strengthens the generalizability of the results of the present example.

Similar to prognostic and predictive biomarkers in active clinical use, the ST-ADT predictive biomarker described herein (e.g., in this example) may be developed and validated as part of a de novo prospective biomarker dedicated trial. This approach is supported by Simon et al, and use of a randomized trial of radiotherapy with or without ADT strengthens the credibility and level of evidence of our work [24]. During the era of conduct and follow-up of this trial, there was effectively no use of advanced molecular imaging. Stage migration due to changes in the Gleason grading system may also have impacted patient stratification into NCCN risk groups. However, any potential biases introduced by this are likely random and impact both arms, and much of the model is driven by the raw histopathology imagery and may not be impacted by changes in definitions of grading over time. Information on other prognostic clinicopathologic variables, such as percentage Gleason pattern 4 or percent positive biopsy cores were not available. Thus, alternative risk-classifications schemas for exploratory analyses were not performed [25, 26].

This example describes a successfully developed and independently validated in a phase III randomized trial predictive biomarker to guide ADT use with radiotherapy in localized prostate cancer using a novel AI-derived digital pathology-based platform. Using this biomarker, the majority of patients may have safely avoided the morbidity and financial toxicity associated with unnecessary ADT treatment.

### REFERENCES

1. Jones, C. U. et al. Adding Short-Term Androgen Deprivation Therapy to Radiation Therapy in Men With Localized Prostate Cancer: Long-Term Update of the NRG/RTOG 9408 Randomized Clinical Trial. Int. J. Radiat. Oncol. Biol. Phys. (2021) doi: 10.1016/j.ijrobp.2021.08.031 is incorporated by reference herein in its entirety.
2. Pilepich, M. V. et al. Androgen suppression adjuvant to definitive radiotherapy in prostate carcinoma—long-term results of phase III RTOG 85-31. Int. J. Radiat. Oncol. Biol. Phys. 61, 1285-1290 (2005) is incorporated by reference herein in its entirety.
3. D'Amico, A. V., Chen, M.-H., Renshaw, A., Loffredo, M. & Kantoff, P. W. Long-term Follow-up of a Randomized Trial of Radiation With or Without Androgen Deprivation Therapy for Localized Prostate Cancer. JAMA vol. 314 1291 (2015) is incorporated by reference herein in its entirety.
4. Bolla, M. et al. Short Androgen Suppression and Radiation Dose Escalation in Prostate Cancer: 12-Year Results of EORTC Trial 22991 in Patients With Localized Intermediate-Risk Disease. J. Clin. Oncol. 39, 3022-3033 (2021) is incorporated by reference herein in its entirety.
5. Kishan, A. U. et al. Androgen deprivation therapy use and duration with definitive radiotherapy for localised prostate cancer: an individual patient data meta-analysis. Lancet Oncol. 23, (2022) is incorporated by reference herein in its entirety.
6. Nguyen, P. L. et al. Adverse effects of androgen deprivation therapy and strategies to mitigate them. Eur. Urol. 67, 825-836 (2015) is incorporated by reference herein in its entirety.
7. Horwitz, E. M. et al. Ten-year follow-up of radiation therapy oncology group protocol 92-02: a phase III trial of the duration of elective androgen deprivation in locally advanced prostate cancer. J. Clin. Oncol. 26, 2497-2504 (2008) is incorporated by reference herein in its entirety.
8. Roach, M., 3rd et al. Phase III trial comparing whole-pelvic versus prostate-only radiotherapy and neoadjuvant versus adjuvant combined androgen suppression: Radiation Therapy Oncology Group 9413. J. Clin. Oncol. 21, 1904-1911 (2003) is incorporated by reference herein in its entirety.
9. Pisansky, T. M. et al. Duration of androgen suppression before radiotherapy for localized prostate cancer: radiation therapy oncology group randomized clinical trial 9910. J. Clin. Oncol. 33, 332-339 (2015) is incorporated by reference herein in its entirety.
10. Jones, C. U. et al. Adding Short-Term Androgen Deprivation Therapy to Radiation Therapy in Men With Localized Prostate Cancer: Long-Term Update of the NRG/RTOG 9408 Randomized Clinical Trial. Int. J. Radiat. Oncol. Biol. Phys. (2021) doi:10.1016/j.ijrobp.2021.08.031 is incorporated by reference herein in its entirety.
11. Michalski, J. M. et al. Effect of Standard vs Dose-Escalated Radiation Therapy for Patients With Intermediate-Risk Prostate Cancer. JAMA Oncology vol. 4 e180039 (2018) is incorporated by reference herein in its entirety.
12. Schaeffer, E. et al. NCCN Guidelines Insights: Prostate Cancer, Version 1.2021: Featured Updates to the NCCN Guidelines. J. Natl. Compr. Canc. Netw. 19, 134-143 (2021) is incorporated by reference herein in its entirety.
13. Tolkach, Y., Dohmgörgen, T., Toma, M. & Kristiansen, G. High-accuracy prostate cancer pathology using deep learning. Nature Machine Intelligence 2, 411-418 (2020) is incorporated by reference herein in its entirety.
14. Nagpal, K. et al. Development and Validation of a Deep Learning Algorithm for Gleason Grading of Prostate Cancer From Biopsy Specimens. JAMA Oncol 6, 1372-1380 (2020) is incorporated by reference herein in its entirety.
15. Pantanowitz, L. et al. An artificial intelligence algorithm for prostate cancer diagnosis in whole slide images of core needle biopsies: a blinded clinical validation and deployment study. Lancet Digit Health 2, e407-e416 (2020) is incorporated by reference herein in its entirety.
16. He, Kaiming et al. Deep Residual Learning for Image Recognition. 2016 IEEE Conference on Computer Vision and Pattern Recognition (CVPR), Jun. 27-30, 2016, ieeexplore.ieee.org/document/7780459 is incorporated by reference herein in its entirety.
17. Chen, X., Fan, H., Girshick, R. & He, K. Improved Baselines with Momentum Contrastive Learning. (2020) is incorporated by reference herein in its entirety.
18. Hutter, F., Kotthoff, L. & Vanschoren, J. Automated Machine Learning: Methods, Systems, Challenges. (Springer, 2019) is incorporated by reference herein in its entirety.
19. Claesen, M. & De Moor, B. Hyperparameter Search in Machine Learning. (2015) doi: 10.48550/arXiv.1502.02127 is incorporated by reference herein in its entirety.
20. Gray, R. J. A Class of K-Sample Tests for Comparing the Cumulative Incidence of a Competing Risk. Ann. Stat. 16, 1141-1154 (1988) is incorporated by reference herein in its entirety.
21. Fine, J. P. & Gray, R. J. A Proportional Hazards Model for the Subdistribution of a Competing Risk. J. Am. Stat. Assoc. 94, 496-509 (1999) is incorporated by reference herein in its entirety.
22. Jones, C. U. et al. Radiotherapy and short-term androgen deprivation for localized prostate cancer. N. Engl. J. Med. 365, (2011) is incorporated by reference herein in its entirety.
23. Spratt, D. E. et al. Development and Validation of a Novel Integrated Clinical-Genomic Risk Group Classification for Localized Prostate Cancer. J. Clin. Oncol. (2017) doi:10.1200/JCO.2017.74.2940 is incorporated by reference herein in its entirety.
24. Simon, R. M., Paik, S. & Hayes, D. F. Use of archived specimens in evaluation of prognostic and predictive biomarkers. J. Natl. Cancer Inst. 101, 1446-1452 (2009) is incorporated by reference herein in its entirety.
25. Cooperberg, M. R. et al. The UCSF Cancer of the Prostate Risk Assessment (CAPRA) Score: a straightforward and reliable preoperative predictor of disease recurrence after radical prostatectomy. J. Urol. 173, 1938 (2005) is incorporated by reference herein in its entirety.
26. A New Risk Classification System for Therapeutic Decision Making with Intermediate-risk Prostate Cancer Patients Undergoing Dose-escalated External-beam Radiation Therapy. Eur. Urol. 64, 895-902 (2013) is incorporated by reference herein in its entirety.
27. Ilse, M., Tomczak, J. & Welling, M. Attention-based Deep Multiple Instance Learning. in Proceedings of the 35th International Conference on Machine Learning (eds. Dy, J. & Krause, A.) vol. 80 2127-2136 (PMLR, 10-15 Jul. 2018) is incorporated by reference herein in its entirety.
28. Austin, P. C. & Stuart, E. A. Moving towards best practice when using inverse probability of treatment weighting (IPTW) using the propensity score to estimate causal treatment effects in observational studies. Stat. Med. 34, 3661-3679 (2015) is incorporated by reference herein in its entirety.
29. Cole, S. R. & Hernán, M. A. Constructing Inverse Probability Weights for Marginal Structural Models. Am. J. Epidemiol. 168, 656-664 (2008) is incorporated by reference herein in its entirety.
30. Lee, B. K., Lessler, J. & Stuart, E. A. Weight Trimming and Propensity Score Weighting. PLOS One 6, e18174 (2011) is incorporated by reference herein in its entirety.
31. Katzman, J. L. et al. DeepSurv: personalized treatment recommender system using a Cox proportional hazards deep neural network. BMC Med. Res. Methodol. 18, 1-12 (2018) is incorporated by reference herein in its entirety.
32. Breslow, N. Covariance analysis of censored survival data. Biometrics 30, 89-99 (1974) is incorporated by reference herein in its entirety.
33. Leen, T. K., Dietterich, T. G. & Tresp, V. Advances in Neural Information Processing Systems 13: Proceedings of the 2000 Conference. (MIT Press, 2001) is incorporated by reference herein in its entirety.
34. Shapley, L. S. 17. A Value for n-Person Games. in Contributions to the Theory of Games (AM-28), Volume II (eds. Kuhn, H. W. & Tucker, A. W.) 307-318 (Princeton University Press, 1953) is incorporated by reference herein in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-33. (canceled)

34. A method for assessing a cancer of a subject, comprising:
(a) obtaining a dataset comprising at least image data obtained or derived from said subject;
(b) processing said dataset using a trained algorithm to determine an output indicative of a classification of said dataset to a category among a plurality of categories, wherein said processing comprises applying an image processing algorithm to said image data; and
(c) assessing said cancer based at least in part on classification of said dataset to said category, wherein said assessing comprises determining a biomarker predictive of a response to a therapeutic intervention for treating said cancer of said subject.

35. The method of claim 34, wherein said response comprises overall survival or progression free survival.

36. The method of claim 34, wherein said response comprises reduction in mortality rate.

37. The method of claim 34, wherein said response comprises metastasis-free survival, reduction in metastasis, or reduction in distant metastasis.

38. The method of claim 34, further comprising determining whether said subject is biomarker positive or biomarker negative for said biomarker, wherein said determining whether said subject is biomarker positive or biomarker negative comprises:
(i) calculating a first probability of said subject displaying said response in a presence of said therapeutic intervention;
(ii) calculating a second probability of said subject displaying said response in an absence of said therapeutic intervention;
(iii) calculating a probability delta between said first probability and said second probability; and
(iv) comparing said probability delta to a reference standard.

39. The method of claim 38, wherein said subject is biomarker positive when said probability delta is higher than said reference standard, and wherein said subject is negative for said biomarker when said probability delta is lower than said reference standard.

40. The method of claim 38, wherein said reference standard is determined at least in part by measuring a median probability delta from a plurality of subjects.

41. The method of claim 38, further comprising treating said subject with said therapeutic intervention.

42. The method of claim 41, wherein said therapeutic intervention comprises androgen deprivation therapy (ADT).

43. The method of claim 42, wherein said ADT is short-term ADT (ST-ADT).

44. The method of claim 34, wherein said trained algorithm comprises self-supervised learning or a deep learning algorithm.

45. The method of claim 34, wherein said dataset further comprises tabular data, and wherein said trained algorithm comprises a first trained algorithm processing said image data and a second trained algorithm processing said tabular data.

46. The method of claim 45, wherein said tabular data comprises clinical data of said subject.

47. The method of claim 46, wherein said clinical data comprises laboratory data, therapeutic interventions, or long-term outcomes.

48. The method of claim 34, wherein said cancer comprises prostate cancer, bladder cancer, breast cancer, pancreatic cancer, or thyroid cancer.

49. The method of claim 48, wherein said cancer comprises prostate cancer.

50. The method of claim 34, wherein said image data comprises digital histopathology data.

51. The method of claim 34, further comprising processing said image data using an image segmentation, image concatenation, or object detection algorithm.

52. A non-transitory computer readable medium comprising machine executable code that, upon execution by one or more computer processors, implements a method for assessing a cancer of a subject, said method comprising:
(a) obtaining a dataset comprising at least image data obtained or derived from said subject;
(b) processing said dataset using a trained algorithm to determine an output indicative of a classification of said dataset to a category among a plurality of categories, wherein said processing comprises applying an image processing algorithm to said image data; and
(c) assessing said cancer based at least in part on classification of said dataset to said category, wherein said assessing comprises determining a biomarker predictive of a response to a therapeutic intervention for treating said cancer of said subject.

53. A system comprising one or more computer processors and computer memory coupled thereto, said computer memory comprising machine executable code that, upon execution by the one or more computer processors, implements a method for assessing a cancer of a subject, said method comprising:
(a) obtaining a dataset comprising at least image data obtained or derived from said subject;
(b) processing said dataset using a trained algorithm to determine an output indicative of a classification of said dataset to a category among a plurality of categories, wherein said processing comprises applying an image processing algorithm to said image data; and
(c) assessing said cancer based at least in part on classification of said dataset to said category, wherein said assessing comprises determining a biomarker predictive of a response to a therapeutic intervention for treating said cancer of said subject.
